(19) United States
(12) Patent Application Publication
(10) Pub. No.: US 2025/0377304 A1
(43) Pub. Date: Dec. 11, 2025

(54) Systems and Methods for Intraoperative Tumor Margin Assessment

(71) Applicant: Marquette University, Milwaukee, WI (US)

(72) Inventors: Bing YU, Gainesville, FL (US); Tongtong LU, Oshkosh, WI (US); Dong Hye YE, Milwaukee, WI (US)

(21) Appl. No.: 19/231,263

(22) Filed: Jun. 6, 2025

Related U.S. Application Data
(60) Provisional application No. 63/656,932, filed on Jun. 6, 2024.

(51) Int. Cl. G01N 21/64; G01N 33/483; G02B 21/16; G02B 21/36; G06T 7/00

(52) U.S. Cl. CPC G01N 21/6458; G01N 21/6428; G01N 33/4833; G02B 21/16; G02B 21/365; G06T 7/0014; G01N 2021/6439; G06T 2207/30096

(57) ABSTRACT

Deep-ultraviolet scanning microscopy uses a first imaging apparatus arranged on a first side of a sample and a second imaging apparatus arranged on a second side of the sample. The first imaging apparatus includes a first ultraviolet light source to illuminate the first side of the sample and a first camera to receive light emitted from the first side of the sample. The second imaging apparatus includes a second ultraviolet light source to illuminate the second side of the sample and a second camera to receive light emitted from the second side of the sample. The first and second sides can be imaged in parallel, and can be sparsely sampled to increase imaging speed. A machine learning model can be used to generate images from the acquired signals. Signals can be detected from intrinsic sources (e.g., tryptophan) and extrinsic sources (e.g., propidium iodide and/or eosin Y) at the same time.

### CROSS-REFERENCE TO RELATED APPLICATIONS

This application claims the benefit of U.S. Provisional Patent Application Ser. No. 63/656,932, filed on Jun. 6, 2024, and entitled “SYSTEMS AND METHODS FOR INTRAOPERATIVE TUMOR MARGIN ASSESSMENT,” which is herein incorporated by reference in its entirety.

### STATEMENT OF FEDERALLY SPONSORED RESEARCH

This invention was made with government support under EB033806 awarded by the National Institutes of Health. The government has certain rights in the invention.

### BACKGROUND

The main goal of cancer surgery is to complete tumor removal while preserving as much normal tissue as possible. Patients with positive margins are at increased risk of recurrence and are recommended to undergo additional surgery, or more toxic treatment (e.g., chemoradiation for oral cancers). Due to an inability to accurately determine margin status during surgery in a timely fashion, a substantial number of patients require additional surgery or treatment. For instance, the current re-excision rate is close to 20% for breast cancer and head and neck squamous cell carcinoma, with significant variation among surgeons. Positive margins and additional surgeries are associated with significant emotional, cosmetic, morbidity, and financial burdens to patients, care providers, and the healthcare system.

### SUMMARY OF THE DISCLOSURE

According to an aspect of the present disclosure, a scanning microscopy system is provided. The scanning microscopy system comprises a sample holder to contain a tissue sample. The scanning microscopy system comprises a first imaging apparatus arranged on a first side of the sample holder, comprising a first ultraviolet light source to illuminate the first side of the sample holder and a first camera to receive light emitted from the tissue sample from the first side of the sample holder. The scanning microscopy system comprises a second imaging apparatus arranged on a second side of the sample holder that is opposite the first side, comprising a second ultraviolet light source to illuminate the second side of the sample holder and a second camera to receive light emitted from the tissue sample from the second side of the sample holder.

According to another aspect of the present disclosure, a method for deep-ultraviolet scanning microscopy is provided. The method comprises acquiring first image data from a sample by illuminating a first side of the sample with a first ultraviolet light source and detecting light emitted from the first side of the sample using a first camera. The method comprises acquiring second image data from the sample by illuminating a second side of the sample with a second ultraviolet light source and detecting light emitted from the second side of the sample using a second camera. The method comprises outputting at least one image of the sample from the first image data and the second image data. Other embodiments of this aspect include corresponding systems (e.g., computer systems, imaging systems), programs, algorithms, and/or modules, each configured to perform the steps of the methods.

According to another aspect of the present disclosure, a method for automated classification of deep ultraviolet fluorescence images for tumor margin assessment is provided. The method comprises dividing a deep ultraviolet fluorescence whole slide image of a tissue specimen into a plurality of patches. The method comprises extracting features from each patch using a first pre-trained convolutional neural network. The method comprises classifying each patch as tumor tissue or normal tissue using a classifier trained on the extracted features. The method comprises generating a regional importance map for the whole slide image using a visual explanation process applied to a second pre-trained convolutional neural network. The method comprises determining a whole slide image classification by fusing patch-level classifications with the regional importance map through a weighted decision fusion. Other embodiments of this aspect include corresponding systems (e.g., computer systems, imaging systems), programs, algorithms, and/or modules, each configured to perform the steps of the methods.

According to another aspect of the present disclosure, a method for semi-automated transfer of tumor annotations from an annotated image to an unannotated image is provided. The method comprises obtaining an annotated image of a tissue specimen captured using a first imaging modality. The method comprises obtaining an unannotated image of the tissue specimen captured using a second imaging modality that is different from the first imaging modality, wherein the annotated image is a different image type than the unannotated image. The method comprises registering the unannotated image to the annotated image using a transformation based on corresponding point pairs selected between the annotated image and the unannotated image. The method comprises extracting tumor annotation outlines from the annotated image. The method comprises refining the extracted annotation outlines by applying edge detection to the registered unannotated image to create a tissue mask and determining an overlap between the annotation outlines and the tissue mask. The method comprises transferring the refined annotation outlines to the registered unannotated image. Other embodiments of this aspect include corresponding systems (e.g., computer systems, imaging systems), programs, algorithms, and/or modules, each configured to perform the steps of the methods.

### BRIEF DESCRIPTION OF THE DRAWINGS

FIGS. 1A and 1B show example deep-ultraviolet scanning microscope (DDSM) systems according to some embodiments described in the present disclosure.

FIG. 2 shows an example of a webcam-assisted selection of an imaging area and start position to be used by a DDSM system.

FIG. 3 is a flowchart of an example method for deep-ultraviolet scanning microscopy and tissue classification.

FIG. 4 is a block diagram of an example system for deep-ultraviolet scanning microscopy.

FIG. 5 is a block diagram of example components that can implement the system of FIG. 4.

FIG. 6 shows an example workflow for classifying a tumor. The WSI are divided into patches to localize cancer detection. With a pre-trained ResNet50 model, the convolutional features are extracted for each patch and used to train an XGBoost classifier for patch-level classification. Grad-CAM++ on a pre-trained DenseNet169 model calculates the regional importance map for the DUV WSI. The patch-level classification results are merged with the regional importance map in a decision fusion for the WSI-level prediction.

FIG. 7A is a block diagram of an example ResNet50 architecture.

FIG. 7B is a block diagram of an example DenseNet169 architecture.

FIG. 8 shows example DUV WSI with corresponding H&E image and regional importance map for malignant and normal/benign samples: Regional importance areas contain high entropy, high content/semantics (3rd column). It focuses on the strong semantic areas to help in the classification. Note that the images are inputs into a pre-trained DenseNet169 with ImageNet weights, and Grad-CAM++ extracted the heatmaps where the relevancy of the tissue areas is determined from 0 to 1. Red and green bounding boxes in DUV WSIs (last column) represent malignant and normal/benign patches thresholded by regional importance, respectively. Large DUV WSIs are scaled down for visualization.

FIG. 9 shows three misclassified DUV WSI normal/benign samples. H&E image and regional importance map are shown for each sample, respectively. Large DUV WSIs are scaled down for visualization.

FIG. 10 illustrates an example workflow for DUV WSI Classification: A WSI is divided into non-overlapping patches individually processed by the vision transformer (ViT). Each patch is further divided into sub-patches, transformed into learnable position and class embeddings, and passed through the transformer encoder. The updated class embedding is then classified via the MLP head. Simultaneously, Grad-CAM++ maps, generated using a fine-tuned CNN, provide patch-level importance weights. Finally, patch-level predictions and Grad-CAM++ weights are fused for the WSI-level classification.

FIG. 11 is a visualization of DUV WSIs with their corresponding H&E images, Grad-CAM++ saliency maps, and Patch-level predictions. Cases (a) and (b) show malignant samples, while (c) and (d) represent benign ones. ViT outperforms CNNs in patch-level predictions, accurately determining patch labels in most cases. While the VIT misclassified some patches in (b) and (c), their impact was mitigated by Grad-CAM++ saliency scores. The proposed method refines WSI classification by heavily weighting diagnostically important regions and de-emphasizing less critical areas.

FIG. 12 depicts an example workflow of a semi-automated annotation process.

FIGS. 13A and 13B illustrate raw MUSE (FIG. 13A) and corresponding H&E (FIG. 13B) images from a breast sample including both malignant and normal tissues.

FIGS. 14A-14C show an example illustrating an image registration process: (FIG. 14A) manually selected points on H&E image; (FIG. 14B) manually selected paired points on MUSE image; (FIG. 14C) registered MUSE image based on the second-order polynomial transformation with coefficients a0=0.1681, a1=0.9862, a2=0.1141, a3=0.0281, a4=−0.0755, a5=−0.1059; b0=0.1227, b1=−0.0214, b2=0.9980, b3=0.0635, b4=−0.1174, b5=0.0190, obtained using the paired points.

FIGS. 15A-15G show an example of annotation extraction and refinement. (FIG. 15A) annotated H&E image; (FIG. 15B) extracted outline from (FIG. 15A); (FIG. 15C) annotation mask created based on (FIG. 15B); (FIG. 15D) registered MUSE image; (FIG. 15E) tissue mask created based on (FIG. 15D); (FIG. 15F) overlap between (FIG. 15C) and (FIG. 15E) (the white area is the overlap and magenta area is the difference); (FIG. 15G) refined annotation.

FIGS. 16A-16C show annotated MUSE images: (FIG. 16A) semi-automatically annotated image; (FIG. 16B) manually annotated raw image; (FIG. 16C) registered manually annotated MUSE image. The comparison was calculated between (FIG. 16A) and (FIG. 16C) with DSC=0.875, cosine similarity=0.977, and CNN similarity=0.986.

FIGS. 17A-17C show examples of manually (first row), semi-automatically (second row) annotated MUSE images, and pathologist-annotated H&E images (third row) of three samples: FIG. 17A, sample with the best performance (Sample #109) with a DSC of 0.96, cosine similarity of 0.92, and CNN scores of 0.94; FIG. 17B, sample with a medium performance (Sample #160) with a DSC of 0.90, cosine similarity of 0.81, and CNN scores of 0.83; and FIG. 17C, sample with the worst similarity (Sample #78) with a DSC of 0.61, cosine similarity of 0.44, and CNN scores of 0.45.

### DETAILED DESCRIPTION

Described here are systems and methods for intraoperative assessment of tumor margins of freshly resected tumor specimens at subcellular resolution and high speed. It is an aspect of the present disclosure to implement a deep-learning enabled, deep-ultraviolet scanning microscope (DDSM) system that can be used to determine the margin status of freshly resected tumor specimens at subcellular resolution within a few minutes. Advantageously, the disclosed DDSM can accurately and efficiently identify positive margins during the initial surgery. Using the disclosed systems and methods, additional tissue can be identified for removal from the surgical cavity until negative margins are achieved, thereby decreasing the need for additional surgery. In this way, unnecessary removal of additional tissue can be avoided.

DDSM uses cost-effective hardware components with a deep-learning based data and/or image analysis to provide high resolution (e.g., 2-3 μm at 4× and 0.5 μm at 20×), large surface coverage (e.g., 10×10 cm), and high speed (e.g., <10 min/specimen) margin assessment. The resulting imaging system has a low-cost, rugged, compact, mobile, easy-to-use system design.

In some aspects, the disclosed systems and methods implement deep ultraviolet (DUV) fluorescence scanning microscopy for simultaneous excitation of multiple fluorophores (e.g., propidium iodide, eosin Y). Additionally, tryptophan imaging can be realized using one or more UV cameras. Advantageously, the disclosed systems and methods can therefore combine intrinsic contrast (e.g., tryptophan) and extrinsic agents (e.g., propidium iodide and eosin Y) for tumor margin detection. Additionally or alternatively, other fluorescent dyes such as rhodamine B, DAPI, Hoechst, acridine orange, and so on, may be used.

Additionally or alternatively, in some aspects the disclosed systems and methods implement parallel imaging to reduce data acquisition time by half by scanning two sides of the sample at the same time.

Microscopy with ultraviolet surface excitation (MUSE) technology represents an approach for real-time imaging of tissue surfaces during surgical procedures. MUSE imaging systems may utilize deep ultraviolet light to excite native tissue fluorophores or extrinsic fluorescent dyes as described herein, thereby generating fluorescence signals that can differentiate between various tissue types based on their biochemical properties.

In surgical oncology, accurate assessment of tumor margins during tissue resection procedures may be beneficial for achieving complete tumor removal while preserving healthy tissue. Traditional intraoperative margin assessment methods, such as frozen section analysis and touch preparation cytology, may involve time-consuming tissue processing steps and may require specialized pathology expertise. MUSE imaging systems may provide an alternative approach by enabling rapid imaging of freshly excised tissue specimens without extensive tissue preparation.

MUSE imaging systems may generate high-resolution fluorescence images that reveal cellular and tissue structures. The fluorescence patterns observed in MUSE images may correspond to different tissue characteristics, allowing for potential differentiation between malignant and normal tissues.

The analysis of MUSE images for tumor margin detection may involve various computational approaches. Texture analysis methods may be applied to extract features from fluorescence images that correlate with tissue types. Machine learning and deep learning algorithms may be trained to classify tissue regions based on these extracted features or direct image analysis.

Annotation of training datasets for machine learning applications in MUSE imaging may present challenges, as pathologists are typically trained to interpret hematoxylin and eosin stained histological sections rather than fluorescence images. Advantageously, the disclosed systems and methods can provide for the transfer of tumor annotations from standard histological images to corresponding MUSE images. This process may involve image registration techniques to account for differences in imaging depth, tissue deformation, and resolution between the two imaging modalities.

Various magnification levels may be employed in MUSE imaging systems, with different magnifications potentially offering trade-offs between imaging speed, field-of-view, and resolution. The selection of appropriate magnification levels may influence the effectiveness of subsequent image analysis algorithms for tumor margin detection.

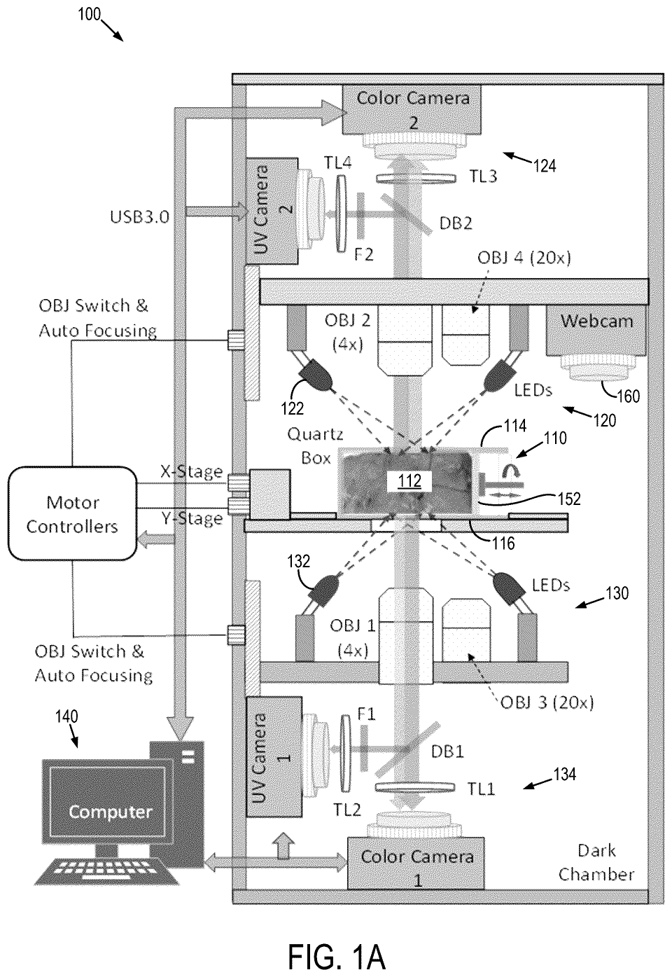

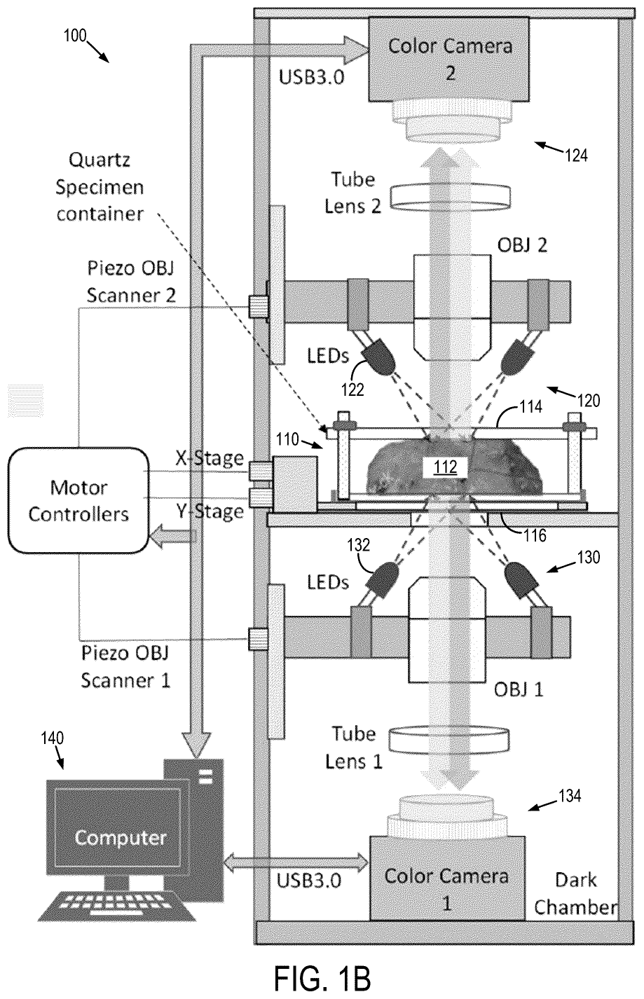

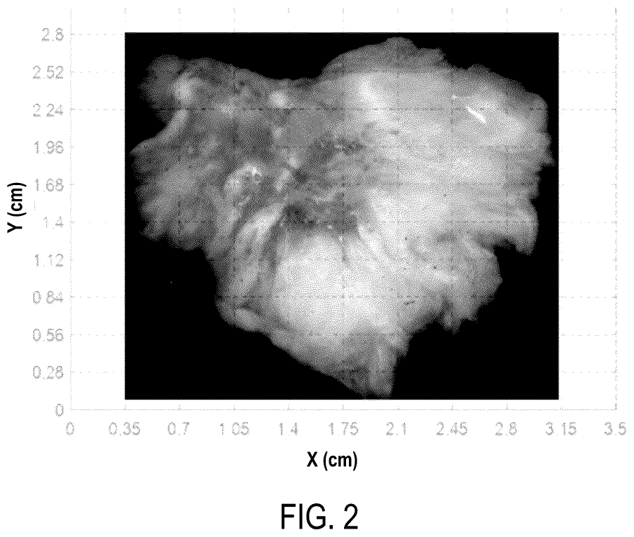

As a non-limiting example, the imaging systems illustrated in FIGS. 1A and 1B can be used to generate sharp, multi-spectral images. In the example illustrated in FIG. 1A, a webcam (or other camera) is also installed next to the objective lenses of the top channel to take a photo of the specimen before starting a new scan. The webcam is installed at a fixed height and distance from the top objective lens and focused on the same plane of the objective lens (i.e., the top surface of a specimen in the illustrated example). The webcam allows the operator to take a photo of the specimen, which can then be used for manual or automated selection of margin areas to scan as described in the present disclosure. Since the relative XY position and heights of the webcam and top objective lens are fixed, the tissue positions in the photo and under the objective lens can be readily co-registered. A photograph of the tissue surface can be input to a machine learning model that has been trained with photos from both malignant and normal breast tissues to automatically select the margin area to be surveyed and first grid (e.g., as shown in FIG. 2) to be used for hotspot searching during sparse sampling.

The imaging systems described in the present disclosure provide a balanced design for subcellular and molecular resolution and rapid imaging of large specimens. In some aspects, this rapid imaging can be achieved by motorized scanning with 13×10 cm travel, autofocus, and specimen handling, and cooled USB 3.0 color and UV cameras. Sparse sampling (SS) may also be used to increase speed. The disclosed imaging systems are also capable of performing coarse scanning with a 4× objective lens for a 2 μm resolution and zoom-in with a 20× objective lens for a 0.5 μm resolution. Advantageously, the disclosed systems and methods allow for visual diagnostic corroboration by the surgeon.

Additionally or alternatively, in some aspects, the disclosed systems and method implement texture analysis and/or deep-learning (DL) algorithms or models for unbiased automated diagnosis.

In some implementations, the disclosed imaging system includes a two-plate quartz specimen holder design for reliable and easy specimen handling. Additionally or alternatively, the disclosed imaging system includes a quartz box design that enables reliable and easy specimen handling by the operator and/or a robotic arm.

Thus, in some aspects, the present disclosure provides a DDSM system that can be used to determine the margin status of freshly resected tumor specimens at subcellular resolution within a few minutes. During the initial surgery, when the DDSM accurately and efficiently identifies positive margins and if anatomically or functionally feasible, additional tissue would be removed from the surgical cavity until negative margins are achieved and unnecessary removal of additional tissue would be avoided, thus decreasing the need for additional surgery. DDSM is a platform technology that can be used for intraoperative margin assessment of multiple cancers (e.g., breast, head & neck, prostate, and skin, etc.), and can also be easily adapted for imaging fresh biopsy specimens and achieving a diagnosis within a few minutes of the procedure.

The deep ultraviolet fluorescence scanning microscope system may utilize a deep UV LED for oblique back illumination to enable fluorescence excitation of tissue samples. In general, deep UV spans a range of 200-300 nm. In some cases, the LED may have a wavelength in a range of 200-300 nm, or a subrange therein. For instance, the LED may generate light with a wavelength in the range of 250-300 nm. As one non-limiting example, the wavelength may be 250 nm. As another non-limiting example, the wavelength may be 285 nm. The system may employ apochromatic long-working-distance objective lenses with different magnifications to accommodate various imaging requirements. In some cases, a 4× objective lens may be used for lower magnification imaging, while an objective lens with a higher magnification can be used for imaging select region with higher spatial resolution. As a non-limiting example, a 20× objective lens may be employed for higher magnification imaging with greater resolution.

The microscope system may include a cooled color camera operated without additional filters for image acquisition. In some cases, the camera may be a cooled USB3.0 color camera with specific sensor specifications tailored for fluorescence imaging applications. The system may incorporate different camera types to accommodate various imaging protocols and requirements.

The illumination system may utilize multiple LED configurations to provide uniform excitation across the tissue surface. In some cases, a single high-power LED may be employed for concentrated illumination. Alternatively, the system may use ring arrangements of low-power LEDs to achieve more uniform illumination distribution across the imaging field.

The microscope system may incorporate a raster scanning mechanism that operates in X and Y directions to ensure coverage of the entire specimen surface. This scanning approach may enable the generation of whole-surface images by capturing individual image tiles from a single margin. The individual image tiles obtained during the scanning process may be computationally aligned and seamlessly stitched together to create comprehensive whole-surface images suitable for analysis. In some aspects, the DDSM system may implement sparse sampling as an additional or alternative data acquisition technique to enhance imaging speed while maintaining diagnostic accuracy. For example, the DDSM system may compress the data acquisition process by acquiring fewer measurements than would be obtained under conventional data acquisition techniques. In some cases, sparse sampling may involve selectively acquiring image data from a subset of locations across the tissue specimen rather than capturing a continuous, high-resolution scan of the entire specimen surface. This approach may significantly reduce overall imaging time, particularly for large tissue samples.

In some cases, the system may provide parallel imaging capability using dual objective lenses to simultaneously image both top and bottom surfaces of tissue specimens. This dual-surface imaging approach may enhance the comprehensive evaluation of tissue margins by providing this simultaneous imaging of both surfaces of the specimen.

Referring again to FIGS. 1A and 1B, an example scanning microscopy system 100 includes a sample holder 110 to contain a tissue sample 112. The sample holder 110 may be configured to securely position and maintain the tissue sample 112 during imaging operations while allowing optical access from multiple directions.

The system 100 includes a first imaging apparatus 120 arranged on a first side 114 of the sample holder 110. The first imaging apparatus 120 includes a first ultraviolet light source 122 to illuminate the first side 114 of the sample holder 110. The first ultraviolet light source 122 may emit deep ultraviolet light at wavelengths suitable for exciting fluorescence in tissue samples, such as approximately 285 nanometers. The first imaging apparatus 120 further includes a first camera 124 to receive light emitted from the tissue sample 112 from the first side 114 of the sample holder 110. The first camera 124 may be configured to capture fluorescence emissions and other optical signals generated by the tissue sample 112 in response to ultraviolet illumination.

The system 100 also includes a second imaging apparatus 130 arranged on a second side 116 of the sample holder 110 that is opposite the first side 114. The second imaging apparatus 130 includes a second ultraviolet light source 132 to illuminate the second side 116 of the sample holder 110. The second ultraviolet light source 132 may operate at similar wavelengths as the first ultraviolet light source 122 to provide consistent illumination conditions. The second imaging apparatus 130 further includes a second camera 134 to receive light emitted from the tissue sample 112 from the second side 116 of the sample holder 110. This dual-sided configuration allows for comprehensive imaging of the tissue sample 112 from multiple perspectives.

In some embodiments, the system 100 further includes a computer system 140 configured to receive first image data from the first camera 124 and second image data from the second camera 134. The computer system 140 may process and analyze the received image data to generate comprehensive imaging results. The computer system 140 is further configured to output one or more images of the sample 112 from the first image data and the second image data. The computer system 140 may perform image processing operations such as stitching, enhancement, and analysis to create composite images or processed representations of the tissue sample 112.

Additionally, the computer system 140 may control the scanning of the XY stages, as described above. In some cases, the computer system 140 may establish communication with the motorized XY stages and perform a calibration routine to ensure accurate positioning. This may involve moving the stages to predefined reference points and verifying position feedback to establish a precise coordinate system for the sample holder 110. The computer system 140 may determine the boundaries and orientation of the tissue sample 112 within the sample holder 110. Based on this information, along with user-defined parameters such as desired resolution and scan area, the computer system 140 generates a scanning pattern. This pattern may take the form of a raster scan, spiral pattern, an adaptive path, or a sparse sampling pattern based on sample features and regions-of-interest, which may be identified in the optical image, for example. The computer system 140 translates the scan plan into a series of movement commands for the XY stages, specifying the direction, speed, and distance of each stage movement. These commands may be synchronized with the activation of the first ultraviolet light source 122 and second ultraviolet light source 132, as well as the image acquisition timing of the first camera 124 and second camera 134. As the XY stages move, the computer system 140 may associate the acquired image data with the corresponding spatial coordinates. This spatial mapping facilitates accurate reconstruction of the whole slide image and enables precise localization of features within the tissue sample 112.

The system 100 may incorporate a zoom-in capability that allows for multi-resolution imaging of the tissue sample 112. This feature enables rapid scanning of large tissue areas while also providing the option for high-resolution examination of specific regions of interest. The system 100 may utilize a 4× objective lens (e.g., as part of the first imaging apparatus 120 and/or the second imaging apparatus 130) to achieve a spatial resolution of approximately 2 μm, which is suitable for initial whole-slide imaging and identification of general tissue architecture and potential areas of concern. For more detailed analysis, the system 100 can seamlessly transition to a higher magnification using a 20× objective lens (e.g., as part of the first imaging apparatus 120 and/or the second imaging apparatus 130), which can provide a refined spatial resolution of approximately 0.5 μm. This zoom-in capability allows for imaging of fine structural details. The computer system 140 may control the objective lens switching mechanism, coordinating the change in magnification with adjustments to the scanning parameters, illumination intensity, and image acquisition settings.

In certain configurations, the sample holder 110 includes an optically transparent box having a moveable plate 152 to compress the sample 112 to fill a volume of the box. The moveable plate 152 may provide controlled compression to ensure proper positioning and flattening of the tissue sample 112 for optimal imaging conditions. The optically transparent box may be composed of quartz, fused silica, or another such material that provides optical transparency for ultraviolet wavelengths while maintaining chemical resistance and structural integrity during imaging operations. Advantageously, using an optically transparent box allows for bulkier tissue specimens to be imaged from multiple sides without having to manually readjust the tissue sample 112 within the sample holder 110.

Additionally or alternatively, the sample holder 110 may include a two-plate design that includes a bottom plate and a top plate, both of which may be composed of optically transparent materials such as quartz to allow for efficient transmission of ultraviolet light and emitted fluorescence signals. The bottom plate of the sample holder 110 may serve as a stable platform on which the tissue sample 112 is placed. In some cases, the bottom plate may feature a slightly recessed area or gentle curvature to help center and contain the tissue sample 112. The top plate can be designed to be lowered onto the tissue sample 112, applying gentle and uniform pressure to flatten both the top and bottom surfaces of the tissue sample 112. This flattening action helps to create a more uniform imaging plane, reducing focus variations and improving overall image quality. The pressure applied by the top plate may be adjustable, allowing for customization based on the specific tissue type and size being examined.

The system 100 may further include an optical camera 160 to acquire an optical image of the tissue sample 112. The optical camera 160 may operate in visible light wavelengths to provide overview imaging capabilities complementary to the ultraviolet fluorescence imaging performed by the first and second imaging apparatus 120, 130.

In embodiments incorporating the optical camera 160, the computer system 140 may be further configured to receive the optical image from the optical camera 160 and determine an imaging area on the tissue sample 112 from the optical image. The computer system 140 may analyze the optical image to identify regions of interest or define scanning boundaries for subsequent detailed imaging. The computer system 140 is configured to direct the first imaging apparatus 120 and second imaging apparatus 130 to acquire first imaging data and second imaging data, respectively, in parallel from the tissue sample 112 by scanning over the determined imaging area. This parallel acquisition capability may enhance imaging efficiency and reduce overall scanning time.

Additionally, the computer system 140 may determine an initial imaging point 170 from the optical image and direct the first imaging apparatus 120 and second imaging apparatus 130 to scan over the determined imaging area starting at the initial imaging point. The initial imaging point may be selected based on tissue characteristics, sample geometry, or other factors to optimize the scanning sequence and ensure comprehensive coverage of the tissue sample 112.

The DDSM system may operate as an integrated platform for intraoperative margin assessment during breast-conserving surgery or other oncological procedures. The system workflow may begin with tissue preparation, where freshly excised surgical specimens are stained with fluorescence dyes such as propidium iodide and eosin Y to enhance contrast between different tissue types. The staining process may take approximately 1-2 minutes and may provide differential fluorescence signals that enable distinction between malignant and normal tissues.

Following staining, the tissue specimen may be positioned on the scanning platform of the DDSM system. The deep UV LED illumination system may provide oblique back illumination for fluorescence excitation across the entire tissue surface. The motorized XYZ stages may enable systematic raster scanning in X and Y directions to capture overlapping image tiles covering the complete specimen surface. In some cases, the scanning process may be completed within 5-10 minutes depending on specimen size and selected magnification.

The image acquisition system may capture individual fluorescence image tiles using either 4× or 10× apochromatic long-working-distance objective lenses with numerical apertures of 0.13 and 0.30 respectively. The cooled color camera may operate without additional filters to collect the fluorescence signals. Each captured image tile may contain spatial information corresponding to a specific region of the tissue specimen surface.

The real-time analysis capabilities of the disclosed systems and methods may enable intraoperative decision-making during breast-conserving surgery procedures. The complete workflow from tissue scanning through image processing to classification results may be completed within 10 minutes, allowing surgeons to assess margin status while the patient remains under anesthesia. The classification results may be presented as color-coded overlay maps on the whole-surface images, with red regions indicating potential tumor areas and green regions indicating normal tissue.

System integration with surgical procedures may involve positioning the scanning platform adjacent to the operating table to minimize tissue transport time and preserve specimen integrity. The fluorescence imaging may be performed on fresh, unprocessed tissue specimens without requiring frozen section preparation or other time-consuming histological processing steps. In some cases, the rapid assessment capabilities may enable immediate re-excision of additional tissue if positive margins are detected, potentially reducing the need for subsequent surgical procedures.

Workflow coordination may involve surgical team members who position specimens on the scanning platform while the primary surgeon continues with other aspects of the procedure. The automated image processing and classification algorithms may operate without requiring specialized technical expertise from surgical personnel. The results display system may provide intuitive visual feedback that enables rapid interpretation by surgeons and pathologists.

Quality control mechanisms may include automatic detection of imaging artifacts, motion blur, or inadequate fluorescence signal intensity that could compromise classification accuracy. The system may provide feedback regarding specimen positioning, staining adequacy, and focus quality to ensure reliable results. In some cases, the system may recommend re-scanning of specific regions or adjustment of imaging parameters to optimize image quality.

A data management system may store all acquired images, processing parameters, and classification results for subsequent review and correlation with final histopathological diagnosis. The integration with hospital information systems may enable automatic patient identification and result documentation. The archived data may serve for continuous algorithm improvement and validation studies comparing intraoperative assessments with definitive histopathological findings.

The image processing pipeline may begin immediately following tile acquisition. Individual image tiles may be computationally aligned and seamlessly stitched using preprocessing algorithms and stitching software to generate whole-surface images. The stitching process may correct for spatial distortions and ensure high-quality, artifact-free images suitable for subsequent analysis. In some cases, the stitched whole-surface images may exceed several gigabytes in file size due to the high resolution and large tissue areas covered.

As a non-limiting example, the image processing workflow may involve raster scanning in X and Y directions to ensure coverage of the entire specimen surface. In some cases, the scanning approach may vary based on the size of the tissue margin being analyzed. For smaller margins having an area of 25 cm² or less, raster scanning may be employed to capture complete coverage of the tissue surface. For larger margins having an area greater than 25 cm², sparse sampling techniques may be utilized to reduce imaging time while maintaining adequate coverage for analysis.

Individual image tiles obtained during the scanning process may be computationally aligned using image registration algorithms. The alignment process may account for potential variations in positioning, rotation, or distortion that may occur during the scanning procedure. Following alignment, the individual tiles may be seamlessly stitched together to form a continuous whole-surface image representing the entire specimen surface.

The preprocessing operations may include image enhancement, noise reduction, and format conversion to prepare the individual tiles for the stitching process. The stitching operations may involve blending algorithms to minimize visible seams between adjacent tiles and ensure smooth transitions across the entire whole-surface image.

The resulting whole-surface images may be divided into smaller patches for subsequent analysis. In some cases, patches having dimensions of 400×400 pixels may be utilized as a standard size for image analysis operations. The patch size may be selected to provide sufficient detail of tissue morphology and cellular structures while maintaining computational efficiency during processing. Different patch sizes may be employed depending on the specific analysis requirements or the magnification level used during image acquisition.

The image processing workflow may generate whole-surface images that are free from stitching artifacts, alignment errors, or other visual distortions that could interfere with subsequent automated analysis or manual interpretation. The processed images may maintain the high contrast and resolution characteristics of the original fluorescence images while providing a comprehensive view of the entire tissue specimen surface.

A texture analysis module may process the stitched whole-surface images by dividing them into smaller patches (e.g., patches of 400×400 pixels). Background regions containing more than a threshold amount of non-tissue areas may in some instances be automatically excluded from analysis to focus computational resources on diagnostically relevant regions. As a non-limiting example, the threshold amount may be 10%, 20%, 30%, 40%, 50%, 60%, 70%, or 80%. A higher threshold amount means that more non-tissue areas will be excluded from subsequent analyses, which thereby reduces computational burden by focusing processing on regions containing substantial tissue. While using a higher threshold decreases processing time and resource requirements, it may also increase the risk of excluding regions containing small but potentially important tissue fragments or features at the specimen margins. Selecting the threshold amount therefore provides a trade-off between computational efficiency against the need for comprehensive tissue analysis. A local binary pattern algorithm may extract texture features from each patch by comparing pixel intensities with neighboring pixels using a uniform rotation-invariant configuration, such as with 12 neighboring pixels at a distance of three pixels from the central pixel.

As a non-limiting example, the texture analysis module may process fluorescence images to extract discriminative features for distinguishing between tumor and normal tissue regions. In some cases, the texture analysis module may employ multiple computational approaches to characterize tissue morphology and cellular patterns within the captured fluorescence images. As noted above, the texture analysis module may divide fluorescence images into smaller patches to enable localized feature extraction and analysis. In some cases, the fluorescence images may be segmented into patches of 400×400 pixels. The patch-based approach may allow the texture analysis module to process high-resolution whole slide images while maintaining computational efficiency and preserving fine-grained tissue details.

A Local Binary Pattern (LBP) method may be implemented in some instances for texture feature extraction from the image patches. The LBP method may compare the intensity of each pixel with neighboring pixels to generate binary patterns that characterize local texture properties. In some cases, the LBP algorithm may examine the intensity relationship between a central pixel and twelve neighboring pixels, forming binary patterns based on whether the neighboring pixels exhibit higher or lower intensity values compared to the central pixel. As an example, a uniform rotation-invariant LBP configuration may be implemented to ensure robustness against rotational variations in tissue orientation. In some cases, the uniform rotation-invariant LBP may maintain consistent texture feature representation regardless of the angular orientation of tissue structures within the fluorescence images. The distance between the central pixel and the neighboring pixels may be set to three pixels to balance computational efficiency with texture representation accuracy.

Additional or alternative texture analysis methods may also be implemented to complement the LBP approach. In some cases, the texture analysis module may employ Grey Level Co-occurrence Matrix (GLCM) analysis to capture spatial relationships between pixel intensities and characterize texture patterns through statistical measures such as contrast, correlation, energy, and homogeneity. The GLCM method may provide complementary texture information that enhances the discriminative power of the overall analysis framework. In this way, the texture analysis module may combine multiple texture analysis methods to create comprehensive feature vectors that capture diverse aspects of tissue morphology and cellular organization. In some cases, the combination of LBP features, GLCM statistics, and nucleus-to-cytoplasmic ratio measurements may provide enhanced discrimination capabilities compared to individual texture analysis approaches. The integrated feature set may improve the accuracy and reliability of tumor margin detection in fluorescence images.

In some implementations, nucleus-to-cytoplasmic ratio analysis may be applied to quantify cellular morphological characteristics that may differentiate between malignant and benign tissue regions. In some cases, the nucleus-to-cytoplasmic ratio analysis may involve segmentation of cellular components within the fluorescence images and calculation of geometric relationships between nuclear and cytoplasmic regions. The ratio measurements may serve as additional features for tissue classification algorithms.

A classification model (e.g., a support vector machine (SVM) classifier with radial basis function (RBF) kernel) may process the extracted texture features to distinguish between normal and tumor tissue patches. The SVM classifier may process the LBP-derived feature vectors to distinguish between normal and tumor tissue patches. In some cases, the SVM classifier may undergo training using cross-validation methodologies to enhance classification performance and generalization capabilities. For example, a ten-fold cross-validation methodology may be implemented, in which 90% of data serves for training and 10% for testing in iterative cycles. In some cases, the texture analysis approach may achieve classification accuracies exceeding 88% for distinguishing tumor from normal tissue.

In some embodiments, a cluster-based decision fusion method focusing on patches with high confident discriminations for margin-level classification may then be used to predict the margin status.

A deep learning classification framework may provide an alternative or complementary analysis pathway. Vision transformer (ViT) models may process image patches to capture both local and global structural dependencies through self-attention mechanisms. The patch-level classification results may be integrated using Grad-CAM++ saliency weighting to highlight spatially relevant regions and enhance diagnostic accuracy. In some cases, the deep learning approach may achieve margin-level classification accuracies exceeding 98% when combining vision transformer models with gradient-based attention mechanisms.

The deep learning classification framework may incorporate multiple neural network architectures and decision fusion methods for analyzing fluorescence images. In some cases, the framework may utilize patch-level processing to handle high-resolution whole slide images that exceed computational memory limitations of direct processing approaches.

A ViT model may serve as a patch-level classifier within the framework. The ViT model may divide input patches into smaller sub-patches that are processed through transformer encoder layers. In some cases, each sub-patch may be flattened into a vector and linearly projected to a fixed dimension through a trainable projection matrix. A trainable class embedding may be concatenated at the start of the sequence to provide an aggregate patch representation. Trainable positional embeddings may be added to preserve spatial location information for each sub-patch within the sequence.

The transformer encoder may process input sequences through multiple layers including layer normalization, multi-head self-attention, and multi-layer perceptron blocks. In some cases, residual connections may be incorporated within both the attention and perceptron blocks. The resulting class token may undergo final layer normalization and pass through a linear classification layer to generate patch-level predictions.

Multiple convolutional neural network architectures may be employed within the classification framework. ResNet50 may serve as a feature extraction backbone in some implementations. DenseNet169 may function as an alternative convolutional architecture for both feature extraction and classification tasks. In some cases, these convolutional networks may be pre-trained on large-scale datasets and fine-tuned for fluorescence image analysis.

Machine learning classifiers may complement the deep learning architectures. An XGBoost classifier may receive features extracted from convolutional networks and perform classification based on gradient boosting algorithms. As described above, an SVM may serve as an alternative classifier that operates on extracted feature representations. In some cases, the SVM may utilize radial basis function kernels for non-linear classification boundaries.

Explainable artificial intelligence techniques may be integrated to generate saliency maps that highlight diagnostically relevant regions. Grad-CAM may compute gradient-based class activation maps by analyzing gradients flowing into convolutional feature maps. Grad-CAM++ may provide enhanced saliency mapping through weighted gradient calculations that isolate positive contributions to class predictions. Layer-wise Relevance Propagation (LRP) may offer an alternative approach for generating relevance scores that trace prediction decisions back through network layers.

The Grad-CAM++ implementation may calculate importance weights for feature maps based on gradient derivatives and weighting coefficients. In some cases, the technique may apply ReLU activation to limit consideration to features with positive contributions to class predictions. Saliency maps generated through Grad-CAM++ may be computed as weighted combinations of feature maps, where weights correspond to calculated importance values. Decision fusion methods may combine patch-level classifications to generate whole slide image predictions. Majority voting may aggregate patch predictions by selecting the class with the highest number of patch votes. Weighted voting may incorporate confidence scores or other weighting factors when combining patch-level decisions. Saliency-based weighting may utilize spatial importance maps to emphasize contributions from diagnostically relevant patches while de-emphasizing less informative regions.

The saliency-based fusion approach may compute patch-level saliency scores by averaging saliency map values over corresponding patch areas. In some cases, patch predictions may be transformed from binary labels to signed values for weighted combination calculations. An empirical threshold may be applied to saliency scores to ensure that only patches with sufficient relevance contribute to the final classification decision. The weighted sum of transformed patch predictions may be processed through a sign function to generate binary whole slide image classifications.

Training procedures may involve fine-tuning pre-trained models on fluorescence image datasets. In some cases, stochastic gradient descent optimization may be employed with cosine learning rate scheduling for ViT model training. Adam optimization may be utilized for convolutional network fine-tuning with specified dropout rates to prevent overfitting. Cross-validation methodologies may be implemented to evaluate model performance across multiple data splits while maintaining separation between training, validation, and test sets.

A semi-automated annotation transfer module may facilitate the creation of training datasets by mapping tumor annotations from pathologist-annotated H&E images to corresponding fluorescence images. The registration process may employ second-order polynomial transformation based on manually selected corresponding points between H&E and fluorescence images. Canny edge detection and morphological refinement may correct annotation outlines and eliminate background inclusion errors. The annotation transfer process may achieve Dice Similarity Coefficients exceeding 0.88 when compared to manual annotations.

The semi-automated annotation transfer method provides a computational approach for mapping tumor annotations from hematoxylin and eosin-stained (H&E) images to corresponding fluorescence images captured using microscopy with ultraviolet surface excitation (MUSE). This method addresses challenges associated with differences in imaging depth, tissue deformation during processing, and variations in contrast and resolution between imaging modalities.

The annotation transfer process may include multiple sequential steps including image registration, outline extraction from H&E images, outline refinement, and application of extracted outlines to fluorescence images. In some cases, the method accommodates geometric distortions and tissue deformations that may occur during formalin-fixed, paraffin-embedded (FFPE) processing.

The image registration component may employ non-rigid registration methods to spatially align H&E and fluorescence images. In some cases, a second-order polynomial transformation may be applied based on manually selected corresponding point pairs between the two imaging modalities. The registration process may accommodate various tissue deformations including stretching, shrinking, folding, or warping that may occur during tissue processing.

The second-order polynomial transformation may be defined by transformation equations that map coordinates from one image space to another. In some cases, at least six pairs of corresponding points may be manually selected from both H&E and fluorescence images to determine transformation coefficients. The transformation may utilize coefficients aᵢ and bᵢ in polynomial equations of the form:

$$X = {a}_{0} + {a}_{1} x + {a}_{2} y + {a}_{3} {x}^{2} + {a}_{4} xy + {a}_{5} {y}^{2}$$

$$Y = {b}_{0} + {b}_{1} x + {b}_{2} y + {b}_{3} {x}^{2} + {b}_{4} xy + {b}_{5} {y}^{2}$$

where (x,y) represent coordinates in the source image and (X,Y) represent corresponding coordinates in the target image.

In some cases, the fluorescence images may be registered to match the spatial configuration of H&E images, which may serve as the reference standard. The registration process may account for differences in penetration depth between imaging modalities, where deep ultraviolet light may have a penetration depth limited to approximately 10 micrometers in biological tissue, while H&E sections may be obtained from cutting depths varying from 0 to 200 micrometers.

Following image registration, tumor annotations from H&E images may be extracted using arithmetic algorithms. In some cases, the extracted annotations may undergo correction using morphological structuring elements to ensure that annotation outlines are closed and enhanced for improved accuracy.

The outline refinement process may incorporate Canny edge detection methods to generate tissue masks. In some cases, Canny edge detection may be applied to identify tissue boundaries and distinguish tissue regions from background areas. The refinement step may involve identifying overlap between annotation masks and tissue masks to eliminate background areas that may have been inadvertently included in manual annotations.

The refined annotation outlines may be obtained by computing the intersection between extracted annotation masks and generated tissue masks. In some cases, this refinement process may reduce annotation errors that may result from manual annotation procedures, such as inclusion of background regions or incomplete outline closure.

Once refined annotation outlines are obtained, the outlines may be transferred to registered fluorescence images. The transfer process may account for differences in image characteristics between H&E and fluorescence imaging modalities, including variations in contrast, appearance, and resolution.

In some cases, the annotation transfer method may accommodate challenges associated with multimodal image registration, including differences in fluorophore characteristics and magnification levels between imaging systems. The method may provide compatibility with various fluorescence imaging configurations and staining protocols.

The semi-automated approach may reduce reliance on manual annotation procedures while maintaining annotation accuracy. In some cases, the method may provide improved consistency compared to manual annotation transfer methods, which may be subject to operator variability and subjective interpretation.

The image registration component may support various registration approaches beyond second-order polynomial transformation. In some cases, alternative non-rigid registration methods may be employed to accommodate different types of tissue deformation or imaging conditions. The registration approach may be selected based on the specific characteristics of the tissue samples and imaging modalities being processed.

In some cases, the registration process may incorporate feature-based matching algorithms or intensity-based registration methods. The selection of registration technique may depend on factors such as tissue type, degree of deformation, and quality of corresponding features between imaging modalities.

The annotation transfer method may provide a framework for generating annotated datasets for training and validation of deep learning classification models. In some cases, the method may facilitate the creation of large-scale annotated fluorescence image datasets that may be used for automated tissue classification and margin assessment applications.

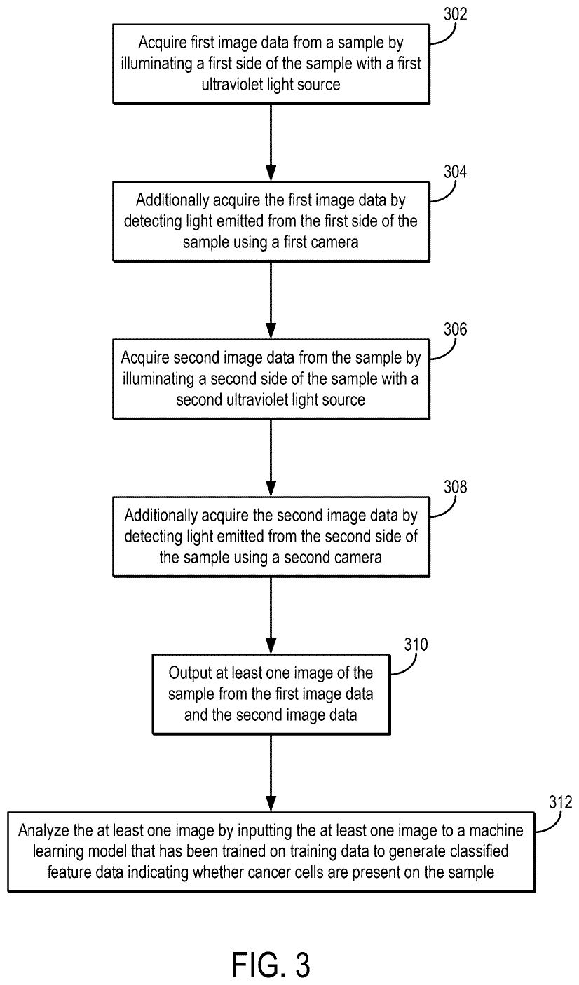

An example method for deep-ultraviolet scanning microscopy will now be described with reference to FIG. 3. At step 302, the method begins by acquiring first image data from a sample by illuminating a first side of the sample with a first ultraviolet light source. The first ultraviolet light source may emit deep ultraviolet light at wavelengths suitable for exciting fluorescence in biological tissues, such as approximately 285 nanometers. The illumination may be provided at an oblique angle or through surface excitation to optimize fluorescence generation while minimizing background interference.

At step 304, the method continues acquiring the first image data by detecting light emitted from the first side of the sample using a first camera. The first camera may be configured to capture fluorescence emissions, scattered light, and other optical signals generated by the sample in response to the ultraviolet illumination. The detected light may include both intrinsic fluorescence from native tissue components and extrinsic fluorescence from applied fluorophores.

At step 306, the method proceeds to acquire second image data from the sample by illuminating a second side of the sample with a second ultraviolet light source. The second side may be positioned opposite to the first side to provide comprehensive illumination coverage of the sample. The second ultraviolet light source may operate at similar wavelengths and intensities as the first ultraviolet light source to ensure consistent imaging conditions across both sides of the sample.

At step 308, the method continues acquiring the second image data by detecting light emitted from the second side of the sample using a second camera. The second camera may have similar specifications and capabilities as the first camera to maintain imaging consistency. The dual-sided detection approach allows for enhanced signal collection and may provide improved contrast and resolution compared to single-sided imaging.

At step 310, the method outputs at least one image of the sample from the first image data and the second image data. The outputting may involve combining, processing, or analyzing the first and second image data to generate composite images, enhanced representations, or processed visualizations of the sample. The output images may be displayed on a monitor, stored in memory, or transmitted to other systems for further analysis.

In some embodiments, the first image data and the second image data include images that include a combination of intrinsic and extrinsic fluorescent signals. The intrinsic fluorescent signals may include fluorescent signals from fluorescent light emitted from tryptophan, which is naturally present in biological tissues and provides characteristic fluorescence patterns when excited by deep ultraviolet light. Other intrinsic fluorophores may include NADH, collagen, elastin, and other native tissue components that exhibit autofluorescence properties.

The extrinsic fluorescent signals may include fluorescent signals from fluorescent light emitted from at least one fluorophore that has been applied to or incorporated into the sample. The at least one fluorophore may include propidium iodide, which is commonly used for nucleic acid staining and provides distinct fluorescence characteristics. Alternatively or additionally, the at least one fluorophore may include eosin Y, which is widely used in histological staining and exhibits specific fluorescence properties under ultraviolet excitation. In certain implementations, the at least one fluorophore includes both propidium iodide and eosin Y, providing complementary staining and fluorescence characteristics for enhanced tissue contrast and differentiation.

The first image data and the second image data may be acquired in parallel, allowing for simultaneous imaging from both sides of the sample. This parallel acquisition approach may reduce overall imaging time and minimize potential artifacts that could arise from temporal variations in sample conditions or environmental factors.

In some implementations, the first image data and the second image data are acquired by sparsely sampling the sample. Sparse sampling may involve capturing images at selected locations or time intervals rather than continuous or dense sampling, which can reduce data acquisition time and computational requirements while maintaining sufficient information for analysis and diagnosis.

At step 312, the method may further include analyzing the at least one image by inputting the at least one image to a machine learning model that has been trained on training data to generate classified feature data indicating whether cancer cells are present on the sample. The machine learning model may include various architectures such as convolutional neural networks, vision transformers, or ensemble methods that have been specifically trained to recognize patterns associated with cancerous tissue. The training data may include annotated images of tissue samples with known pathological classifications, allowing the model to learn distinguishing features between malignant and normal tissue. The classified feature data may provide quantitative assessments, probability scores, or binary classifications indicating the presence or absence of cancer cells in specific regions of the sample.

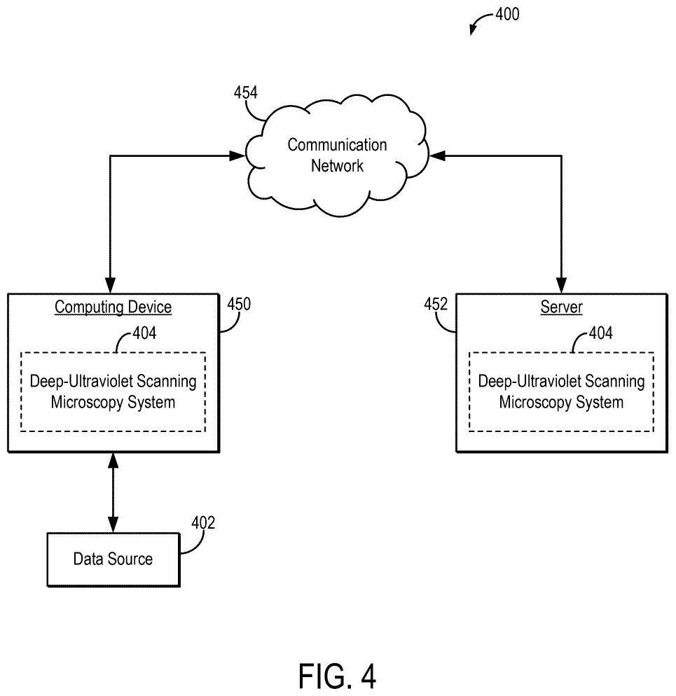

FIG. 4 shows an example of a system 400 for deep-learning based ultraviolet scanning microscopy in accordance with some embodiments described in the present disclosure. As shown in FIG. 4, a computing device 450 can receive one or more types of data (e.g., imaging data) from data source 402. In some embodiments, computing device 450 can execute at least a portion of a deep-ultraviolet scanning microscopy (DDSM) system 404 to generate images from data received from the data source 402.

Additionally or alternatively, in some embodiments, the computing device 450 can communicate information about data received from the data source 402 to a server 452 over a communication network 454, which can execute at least a portion of the DDSM system 404. In such embodiments, the server 452 can return information to the computing device 450 (and/or any other suitable computing device) indicative of an output of the DDSM system 404.

In some embodiments, computing device 450 and/or server 452 can be any suitable computing device or combination of devices, such as a desktop computer, a laptop computer, a smartphone, a tablet computer, a wearable computer, a server computer, a virtual machine being executed by a physical computing device, and so on. The computing device 450 and/or server 452 can also reconstruct images from the data.

In some embodiments, data source 402 can be any suitable source of data (e.g., measurement data, images reconstructed from measurement data, processed image data), such as an ultraviolet scanning microscopy system (e.g., the system illustrated in FIG. 1), another computing device (e.g., a server storing measurement data, images reconstructed from measurement data, processed image data), and so on. In some embodiments, data source 402 can be local to computing device 450. For example, data source 402 can be incorporated with computing device 450 (e.g., computing device 450 can be configured as part of a device for measuring, recording, estimating, acquiring, or otherwise collecting or storing data). As another example, data source 402 can be connected to computing device 450 by a cable, a direct wireless link, and so on. Additionally or alternatively, in some embodiments, data source 402 can be located locally and/or remotely from computing device 450 and can communicate data to computing device 450 (and/or server 452) via a communication network (e.g., communication network 454).

In some embodiments, communication network 454 can be any suitable communication network or combination of communication networks. For example, communication network 454 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 454 can be a local area network, a wide area network, a public network (e.g., the Internet), a private or semi-private network (e.g., a corporate or university intranet), any other suitable type of network, or any suitable combination of networks. Communications links shown in FIG. 4 can each be any suitable communications link or combination of communications links, such as wired links, fiber optic links, Wi-Fi links, Bluetooth links, cellular links, and so on.

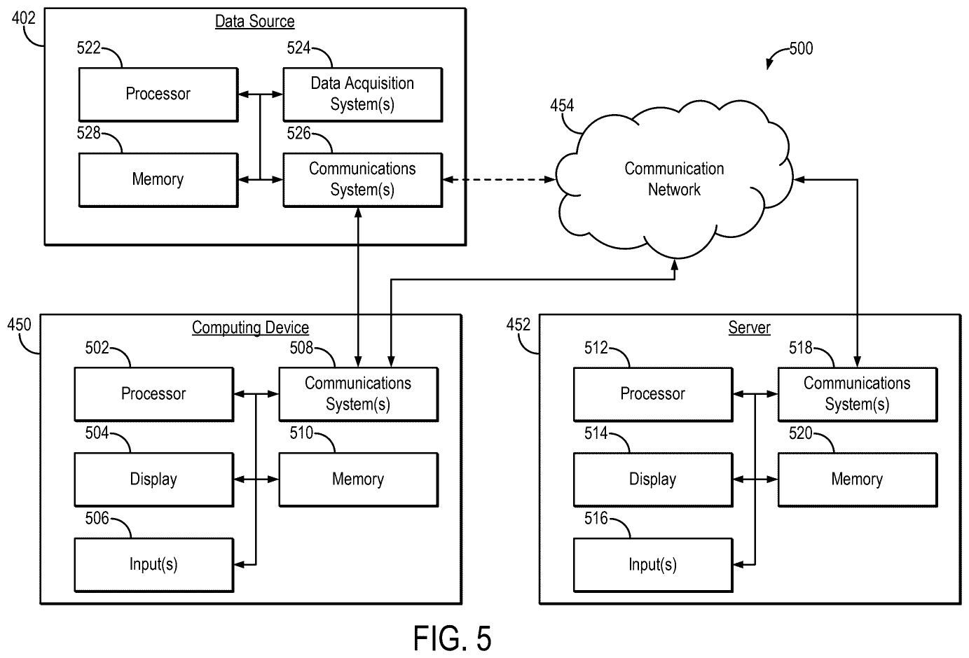

Referring now to FIG. 5, an example of hardware 500 that can be used to implement data source 402, computing device 450, and server 452 in accordance with some embodiments of the systems and methods described in the present disclosure is shown.

As shown in FIG. 5, in some embodiments, computing device 450 can include a processor 502, a display 504, one or more inputs 506, one or more communication systems 508, and/or memory 510. In some embodiments, processor 502 can be any suitable hardware processor or combination of processors, such as a central processing unit (“CPU”), a graphics processing unit (“GPU”), and so on. In some embodiments, display 504 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 506 can include any suitable input devices and/or sensors that can be used to receive user input, such as a keyboard, a mouse, a touchscreen, a microphone, and so on.

In some embodiments, communications systems 508 can include any suitable hardware, firmware, and/or software for communicating information over communication network 454 and/or any other suitable communication networks. For example, communications systems 508 can include one or more transceivers, one or more communication chips and/or chip sets, and so on. In a more particular example, communications systems 508 can include hardware, firmware, and/or software that can be used to establish a Wi-Fi connection, a Bluetooth connection, a cellular connection, an Ethernet connection, and so on.

In some embodiments, memory 510 can include any suitable storage device or devices that can be used to store instructions, values, data, or the like, that can be used, for example, by processor 502 to present content using display 504, to communicate with server 452 via communications system(s) 508, and so on. Memory 510 can include any suitable volatile memory, non-volatile memory, storage, or any suitable combination thereof. For example, memory 510 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 510 can have encoded thereon, or otherwise stored therein, a computer program for controlling operation of computing device 450. In such embodiments, processor 502 can execute at least a portion of the computer program to present content (e.g., images, user interfaces, graphics, tables), receive content from server 452, transmit information to server 452, and so on. For example, the processor 502 and the memory 510 can be configured to perform the methods described herein.

In some embodiments, server 452 can include a processor 512, a display 514, one or more inputs 516, one or more communications systems 518, and/or memory 520. In some embodiments, processor 512 can be any suitable hardware processor or combination of processors, such as a CPU, a GPU, and so on. In some embodiments, display 514 can include any suitable display devices, such as an LCD screen, LED display, OLED display, electrophoretic display, a computer monitor, a touchscreen, a television, and so on. In some embodiments, inputs 516 can include any suitable input devices and/or sensors that can be used to receive user input, such as a keyboard, a mouse, a touchscreen, a microphone, and so on.

In some embodiments, communications systems 518 can include any suitable hardware, firmware, and/or software for communicating information over communication network 454 and/or any other suitable communication networks. For example, communications systems 518 can include one or more transceivers, one or more communication chips and/or chip sets, and so on. In a more particular example, communications systems 518 can include hardware, firmware, and/or software that can be used to establish a Wi-Fi connection, a Bluetooth connection, a cellular connection, an Ethernet connection, and so on.

In some embodiments, memory 520 can include any suitable storage device or devices that can be used to store instructions, values, data, or the like, that can be used, for example, by processor 512 to present content using display 514, to communicate with one or more computing devices 450, and so on. Memory 520 can include any suitable volatile memory, non-volatile memory, storage, or any suitable combination thereof. For example, memory 520 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 520 can have encoded thereon a server program for controlling operation of server 452. In such embodiments, processor 512 can execute at least a portion of the server program to transmit information and/or content (e.g., data, images, a user interface) to one or more computing devices 450, receive information and/or content from one or more computing devices 450, receive instructions from one or more devices (e.g., a personal computer, a laptop computer, a tablet computer, a smartphone), and so on.

In some embodiments, the server 452 is configured to perform the methods described in the present disclosure. For example, the processor 512 and memory 520 can be configured to perform the methods described herein.

In some embodiments, data source 402 can include a processor 522, one or more data acquisition systems 524, one or more communications systems 526, and/or memory 528. In some embodiments, processor 522 can be any suitable hardware processor or combination of processors, such as a CPU, a GPU, and so on. In some embodiments, the one or more data acquisition systems 524 are generally configured to acquire data, images, or both, and can include an ultraviolet microscopy system (e.g., the system shown in FIG. 1). Additionally or alternatively, in some embodiments, the one or more data acquisition systems 524 can include any suitable hardware, firmware, and/or software for coupling to and/or controlling operations of an ultraviolet microscopy system (e.g., the system shown in FIG. 1). In some embodiments, one or more portions of the data acquisition system(s) 524 can be removable and/or replaceable.

Note that, although not shown, data source 402 can include any suitable inputs and/or outputs. For example, data source 402 can include input devices and/or sensors that can be used to receive user input, such as a keyboard, a mouse, a touchscreen, a microphone, a trackpad, a trackball, and so on. As another example, data source 402 can include any suitable display devices, such as an LCD screen, an LED display, an OLED display, an electrophoretic display, a computer monitor, a touchscreen, a television, etc., one or more speakers, and so on.

In some embodiments, communications systems 526 can include any suitable hardware, firmware, and/or software for communicating information to computing device 450 (and, in some embodiments, over communication network 454 and/or any other suitable communication networks). For example, communications systems 526 can include one or more transceivers, one or more communication chips and/or chip sets, and so on. In a more particular example, communications systems 526 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 528 can include any suitable storage device or devices that can be used to store instructions, values, data, or the like, that can be used, for example, by processor 522 to control the one or more data acquisition systems 524, and/or receive data from the one or more data acquisition systems 524; to generate images from data; present content (e.g., data, images, a user interface) using a display; communicate with one or more computing devices 450; and so on. Memory 528 can include any suitable volatile memory, non-volatile memory, storage, or any suitable combination thereof. For example, memory 528 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 528 can have encoded thereon, or otherwise stored therein, a program for controlling operation of data source 402. In such embodiments, processor 522 can execute at least a portion of the program to generate images, transmit information and/or content (e.g., data, images, a user interface) to one or more computing devices 450, receive information and/or content from one or more computing devices 450, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

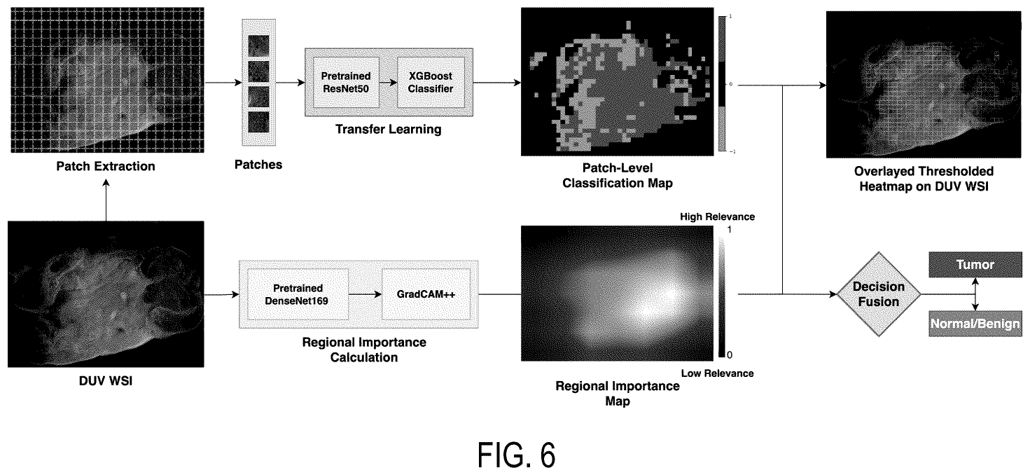

In an example study, the performance accuracy of the proposed approach was tested by classifying 60 images at the WSI level. FIG. 6 describes the proposed breast cancer classification method for DUV image margin assessment. First, DUV WSIs are divided into patches to enhance the dataset and localize cancerous regions. A pre-trained ResNet50 model extracts convolutional features for each patch, which are then used to train an XGBoost classifier for patch-level classification. The regional importance map for the DUV WSI is calculated using Grad-CAM++ on a pre-trained DenseNet169 model. Finally, the patch-level classification results merge with the regional importance map in a decision fusion for the WSI-level prediction.

A DUV WSI is divided into multiple DUV patches. The xi is denoted as a DUV WSI for a sample i. A 2D grid system is designed to be based on each sample's field of view (FOV) Ωᵢ with non-overlapping patches

$${Ω}_{i}^{j} ,$$

$${Ω}_{i} = {⋃}_{j = 1} N {Ω}_{i}^{j} and {Ω}_{i}^{k} ⋂ {Ω}_{i}^{l} = ∅ for ∀ k , l$$

where N represents the total amount of patches in xᵢ, and k and l represent any patch indices.

Since each DUV WSI has different dimensions, the images are resized to the closest dimensions divisible into non-overlapping patches

$${Ω}_{i}^{j}$$

with a constraint size of 400×400 pixels.

The minimal alterations to the dimensions should not affect the quality of the morphological characteristics, such as cell density and infiltration, because the DUV WSI images are very large. Afterward, a 2D grid system is implemented based on each sample's FOV Ωᵢ with non-overlapping cells

$${Ω}_{i}^{j} .$$

The patch images are extracted from each non-overlapping patch

$${Ω}_{i}^{j}$$

of a DUV WSI. To determine the validity of a patch, it is converted to grayscale, and its pixels are analyzed. If most of the pixels in the patch

$${Ω}_{i}^{j}$$

are at least 80% foreground, then it is counted as a valid patch,

$${p}_{i}^{j} .$$

A pixel is a foreground it its grayscale value was greater than 5. It is important to remove the dark background to help with the localization and detection of breast cancer. This process is shown in Algorithm 1 ExtractPatches, and Algorithm 2 GetPercentage.

Algorithm 1: ExtractPatches is used to extract foreground
patches for each DUV WSI

| xᵢ | DUV WSI sample i |
|---|---|
| Ωᵢ | 2D grid system on each sample's FOV |
| ${Ω}_{i}^{j}$ | Non- overlapping patch with 400 × 400 pixels |
| ${p}_{i}^{j}$ | Valid patch image |
| M | Number of DUV WSI samples |
| for i < M do | |
| | Load in sample x; |
| | $for {Ω}_{i}^{j} in {Ω}_{i} do$ |
| | $if GetPercentage ( {Ω}_{i}^{j} ) \geq 0.2 then$ |
| | $Save {Ω}_{i}^{j} as valid patch {p}_{i}^{j} for {Ω}_{i}$ |
| | end if |
| | end for |

end for

Algorithm 2: GetPercentage is used to determine
the foreground percentage of a patch

| BackgroundThreshold5 | | |
|---|---|---|
| ImageSize | 400 × 400 pixels | |
| PixelCounter | ← | 0 |

for x < 400 do

for y < 400 do

if Image[x][y] ≥ BackgroundThreshold then

PixelCounter ← PixelCounter + 1

end if

end for

end for

return PixelCounter = ImageSize

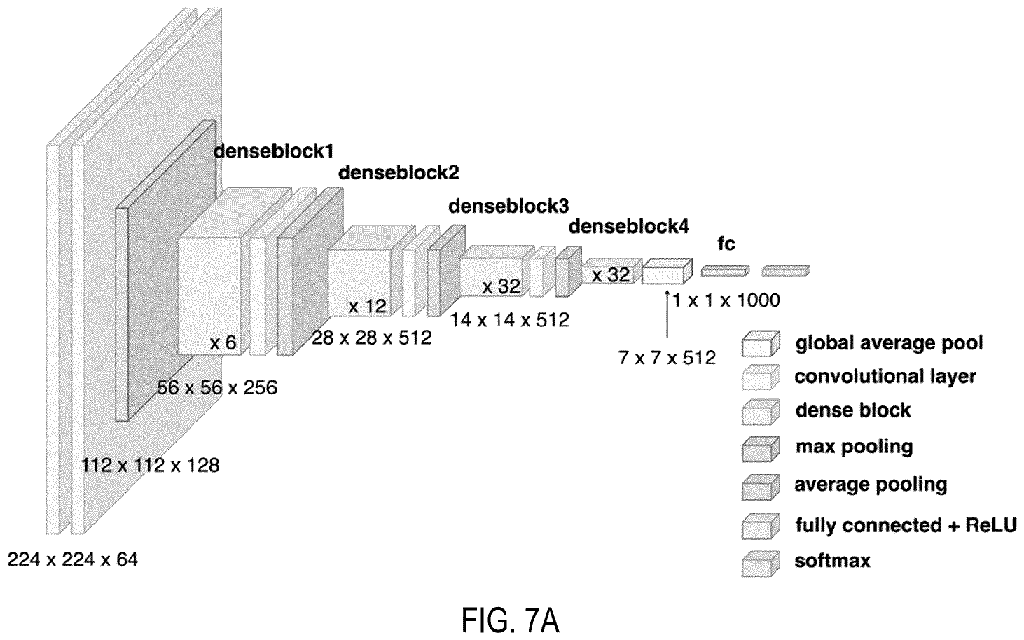

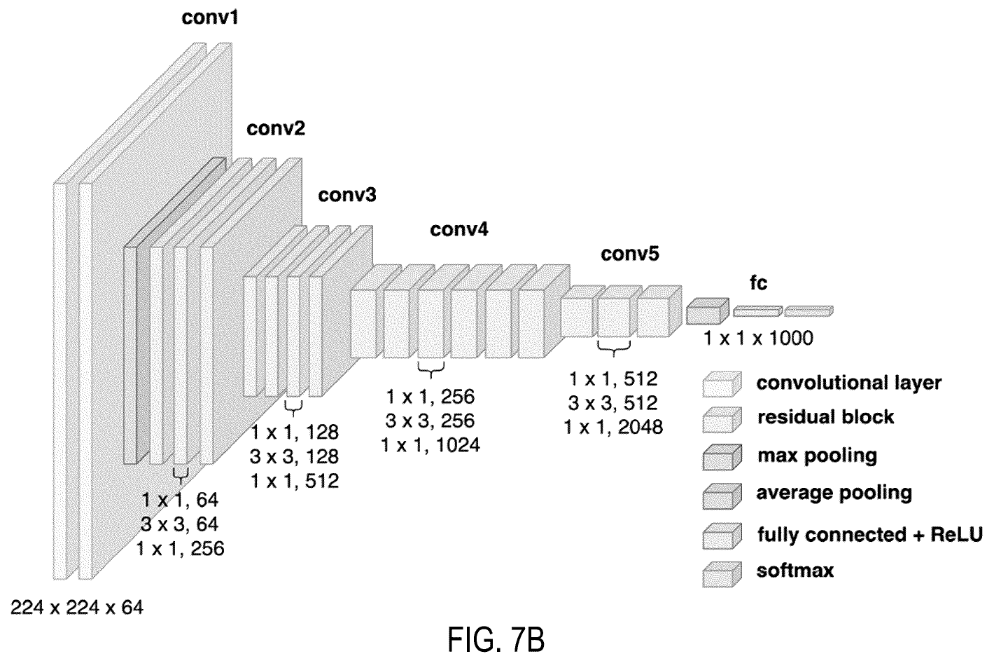

Convolutional neural networks are widely used for image classification problems and have the potential to diagnose diseases. In architectures like ResNet, the ‘vanishing gradient’ problem exists. The architecture of an example ResNet50 is visualized in FIG. 7A. This happens as the increased number of layers can cause information loss of certain/specific information, preventing the network from training well. Information loss is a critical problem to overcome, especially with training with the limited DUV dataset. Even though the gradient of ResNets can transfer directly to the identity function from the previous layers to the forward layers, the summation of the identity function and output H can nullify the information flow in the network. A robust model architecture is needed to prevent overfitting for a small BCS dataset.

For this issue, a transfer learning approach determines whether each patch is malignant or normal/benign for all N DUV patches

$${p}_{i}^{j} ,$$

with j={1, . . . , N}. In this approach, the features are extracted from the final layer of a pre-trained ResNet50 network on the ImageNet dataset and fed into an XGBoost classifier. The XGboost classifier predicts the binary output

$${y}_{i}^{j} ∈ \{ + 1 , - 1 \}$$

as malignant or normal/benign for each patch

$${p}_{i}^{j} .$$

This determines the tumor ROI in the DUV WSI based on

$${y}_{i}^{j}$$

with the relative paten locations, as depicted in FIG. 6.

Grad-CAM++ is an explainable artificial intelligence approach that generates visual explanations behind a model's decisions. When applied to deep neural networks, it can visualize gradients with pixel-wise weighted feature maps. This technique explains the output layer decisions while considering the spatial information and high-level semantics from the previous convolutional layers. Since the details in the patches and the DUV images are very complex, it is crucial to implement Grad-CAM++ on a network that can retain as much information as it can. A proposed network called DenseNet (Densely Connected Convolutional Network) is a modified version of ResNet where each layer is connected directly with every other layer.

As shown in this equation, the Ith layer receives the feature-maps of all preceding layers, X₀, . . . , X₍ₗ₋₁₎, as input,

$${x}_{l} = {H}_{l} ( [ {x}_{0} , … , {x}_{( l - 1 )} ] )$$

where [x₀, . . . , x₍ₗ₋₁₎] refers to the concatenation of the feature maps produced in layers 0 to (l−1).

Advantageously, DenseNet removes repeated feature maps after the concatenation of the feature maps, allowing for fewer parameters. By concatenating these feature maps from different feature maps, it increases the variation in the inputs of the following layers. The bottleneck and compression layers of the DenseNet architecture are effective against overfitting due to fewer parameters needed. The bottleneck layers help reduce the number of inputs from previous k-output feature maps, which improves computational efficiency. For the compression layers, they improve model compactness by reducing the feature maps that a dense block generates. The feature maps will overlap at the last output layer, highlighting the most relevant features, which is helpful for Grad-CAM++.

The following equations describe Grad-CAM++'s process of extracting weight maps from feature maps. The final classification score is defined as Y^{c} for class c as a linear combination of global average pooled convolutional feature maps A^{k} in the last layer over the FOV Ωᵢ:

$${Y}^{c} = {\sum }_{k} {w}_{k}^{c} \cdot {\sum }_{l ∈ {Ω}_{i}} {A}_{l}^{k} .$$

The gradient of the classification score Y^{c} for class c before the softmax layer with respect to the final convolutional layer feature map activation A^{k} is defined as

$$\frac{\partial {Y}^{c}}{\partial {A}^{k}} .$$

The weights

$${w}_{k}^{c}$$

for a particular feature map A^{k} are defined by its average pooled gradients:

$${w}_{k}^{c} = \frac{1}{Z} {\sum }_{l ∈ {Ω}_{i}} \frac{\partial {Y}^{c}}{\partial {A}_{l}^{k}}$$

where Z represents the activation map's number of pixels.

With Grad-CAM implementations, visualizations are limited if there are multiple instances of a class in the input image xᵢ, as different spatial footprints of classes can cause different feature maps. The feature maps with small footprints will not be seen in the final saliency map. To fix this issue, a weighted average of the pixel-wise gradients can be taken:

$${w}_{k}^{c} = {\sum }_{l ∈ {Ω}_{i}} {α}_{l}^{kc} \cdot ReLU ( \frac{\partial {Y}^{c}}{\partial {A}_{l}^{k}} )$$

where ReLU is the rectified linear unit activation function and

$${α}_{l}^{kc}$$

correspond to the pixel-wise gradients for class c and convolutional feature map A^{k}. The gradient weights

$${α}_{l}^{kc}$$

can be derived for a particular class c and activation map A^{k}:

$${α}_{l}^{kc} = \frac{\frac{{\partial }^{2} {Y}^{c}}{{( \partial {A}_{l}^{k} )}^{2}}}{2 \frac{{\partial }^{2} {Y}^{c}}{{( \partial {A}_{l}^{k} )}^{2}} + {\sum }_{l ∈ {Ω}_{i}} {A}_{l}^{k} \frac{{\partial }^{3} {Y}^{c}}{{( \partial {A}_{l}^{k} )}^{3}}}$$

The Grad-CAM++ weights are calculated as:

$${w}_{k}^{c} = {\sum }_{l ∈ {Ω}_{i}} [ \frac{\frac{{\partial }^{2} {Y}^{c}}{{( \partial {A}_{l}^{k} )}^{2}}}{2 \frac{{\partial }^{2} {Y}^{c}}{{( \partial {A}_{l}^{k} )}^{2}} + {\sum }_{l ∈ {Ω}_{i}} {A}_{l}^{k} \frac{{\partial }^{3} {Y}^{c}}{{( \partial {A}_{l}^{k} )}^{3}}} ] \cdot relu ( \frac{\partial {Y}^{c}}{\partial {A}_{l}^{k}} )$$

The regional importance map Rⁱ for a DUV WSI xᵢ is computed using a linear combination of forward activation maps:

$${R}^{i} = ReLU ( {\sum }_{k} {w}_{k}^{c} \cdot {A}_{l}^{k} ) .$$

This highlights the most significant features in the final classification with a positive correlation with pixel intensity and classification score with the application of the ReLU function to a linear combination of activation maps. In this study, the GradCAM++ implementation is applied on a pre-trained DenseNet169 model with ImageNet weights and extracts the feature map as the regional importance map at the Norm5 layer. The architecture of the DenseNet169 model can be visualized in FIG. 7B.

Given the patch-level classification labels

$${y}_{i}^{j} ∈ \{ + 1 , - 1 \}$$

for all patches j={1, . . . , Mᵢ} and regional importance map Rⁱ, a decision fusion method is applied to determine the WSI-level classification label yᵢ={+1, −1}. First, the regional importance

$${r}_{i}^{j}$$

is computed for each patch

$${p}_{i}^{j}$$

by taking the average value of Rⁱ over a patch's FOV

$${Ω}_{i}^{j} :$$

$${r}_{i}^{j} = \frac{1}{\vert {Ω}_{i}^{j} \vert} {\sum }_{l ∈ {Ω}_{i}^{j}} {R}_{l}^{j}$$

where

$$\vert {Ω}_{i}^{j} \vert$$

is the number of pixels for each patch (e.g., 400×400 pixels).

The weight is defined as

$${w}_{i}^{j}$$

for each patch

$${p}_{i}^{j}$$

based on the thresholded regional importance value

$${r}_{i}^{j} :$$

$${w}_{i}^{j} = \{ \begin{matrix}0 & if {r}_{i}^{j} < 0.25 \\ {r}_{i}^{j} & otherwise\end{matrix} .$$

This weighting scheme ignores the patches with low importance, either malignant or normal/benign, in the fused decision for the DUV WSI.

Next, the patch-level classification label

$${y}_{i}^{j} and {w}_{i}^{j}$$

are multiplied for each patch as

$${u}_{i}^{j} :$$

$${u}_{i}^{j} = {w}_{i}^{j} \cdot {y}_{i}^{j} .$$

Now, the total amount of malignant patches Hᵢ is calculated,

$${H}_{i} = \{ \begin{matrix}1 & if {u}_{i}^{j} > 0 \\ 0 & otherwise\end{matrix} .$$

Finally, the WSI-level classification label yᵢ is determined by comparing Hᵢ to a certain percentage q of the total foreground patches Mᵢ:

$${y}_{i} = \{ \begin{matrix}+ 1 & if {H}_{i} > q \cdot {M}_{i} , 0 \leq q \leq 1 \\ - 1 & otherwise\end{matrix}$$

where the positive (malignant) and negative (benign) values are mapped to +1 and −1, respectively.

Automated DUV WSI classification is used to determine if a sample is malignant or normal/benign tissue. The proposed method was assessed with 5-fold cross-validation. The DUV WSI data was split while managing balanced class labels and specific patient samples to stay only in training or testing data splits. There was no fine-tuning of the pre-trained ResNet50 with ImageNet weights for the Transfer Learning part. The XGBoost classifier's hyperparameters were default settings and did not involve any tinkering. There was no hyperparameter tuning for the pre-trained DenseNet169 model with ImageNet Weights for the Regional Importance Calculation. The proposed approach was compared with a standard ResNet50 model and Patch Classification with Majority Voting. The ResNet50 (17) trained for 100 epochs on the limited DUV WSI data with a batch size of 4, a learning rate of 0.006, and a dropout of 40%. Hyperparameter tuning has been done for the ResNet50 model. The Patch Classification with Majority Voting is derived from the architecture of the proposed method's patch classification and transfer learning portion. This approach is augmented with a patch majority voting scheme for binary classification between malignant and normal/benign for the WSIs.

The breast cancer dataset included DUV images from 60 samples (24 normal/benign and 36 malignant). The DUV-FSM used a deep ultraviolet (DUV) excitation at 285 nm and a low magnification objective (4×), which achieved a small spatial resolution from 2 to 3 mm. To enhance fluorescence contrast, breast tissues are stained with propidium iodide and eosin Y. This technique produces images of the microscopic resolution, sharpness, and contrast from fresh tissue stained with multiple fluorescence dyes.

The 60 DUV images were divided into 34468 patches with a size of 400×400 pixels at 4× magnification (9444 malignant and 25024 normal/benign samples). Moreover, when the classifiers were trained, horizontal/vertical flips and 90-degree rotations were used to boost the data. The pathologists annotated and delineated tumors from the corresponding H&E images for ground-truth labels. The DUV images are registered and compared with H&E images manually.

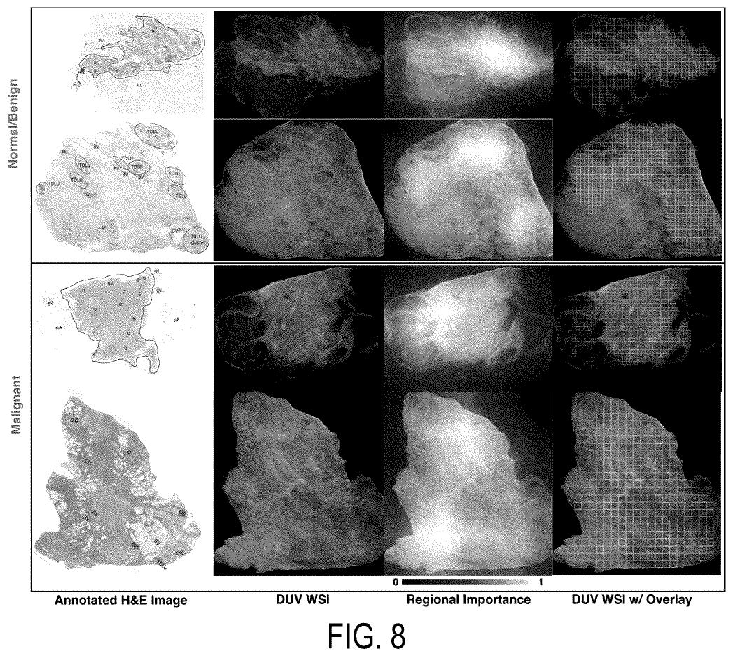

The breast cancer location and detection results on several malignant and normal/benign DUV images for qualitative evaluation are in the last column of FIG. 8. The Grad-CAM++ influenced results are shown as the red (malignant) and green (benign) patches with high importance. The H&E images annotated by the pathologists are displayed for comparison with their DUV counterparts. In comparison, the DUV images have a higher color contrast than the H&E images when analyzing the malignant (pink/yellow) and normal/benign (light/dark green) tissues.

As seen in these DUV WSI images, the proposed method was able to accurately localize malignant and normal/benign areas with the aid of Grad-CAM++ regional importance maps. The DUV images contain overlaid regions focusing on malignant (red squares) and normal/benign adipose (green) tissue. These DUV images show that the proposed fusion method with regional importance maps can localize ROI for a WSI-level decision with confident margin prediction accuracy. The results demonstrate that even with little training data, this deep learning approach with a patching strategy can capture pathological traits like high cell density and infiltration.

The accuracy, sensitivity, specificity, and AUC score of malignant/benign binary classification on DUV-WSI images are measured for quantitative analysis. Compared to standard ResNet50, the proposed method significantly improves classification performance with a 13.3% increase in accuracy. The proposed method is reliable, while the standard ResNet50 with DUV WSI has overfitting issues. The proposed method outperforms ResNet50 in sensitivity, specificity, and AUC scores by 8.3%, 20.8%, and 17.0%, respectively. Compared with Patch Classification with Majority Voting, the proposed method outperforms by 1.7% and 5.6% for accuracy and sensitivity, respectively. Although the proposed method has a specificity that is 3.7% lower than another approach, it is still a good option for breast cancer classification. The proposed method achieved a perfect sensitivity rate of 100% compared to the other approach. Sensitivity is crucial in detecting malignant cases, a primary concern in breast cancer classification. The higher sensitivity rate of the proposed method makes it a valuable option despite having slightly lower specificity. These metrics demonstrate the advantage of the proposed method for intra-operative margin assessment, as it should reduce the likelihood of breast cancer margins being undetected during BCS.

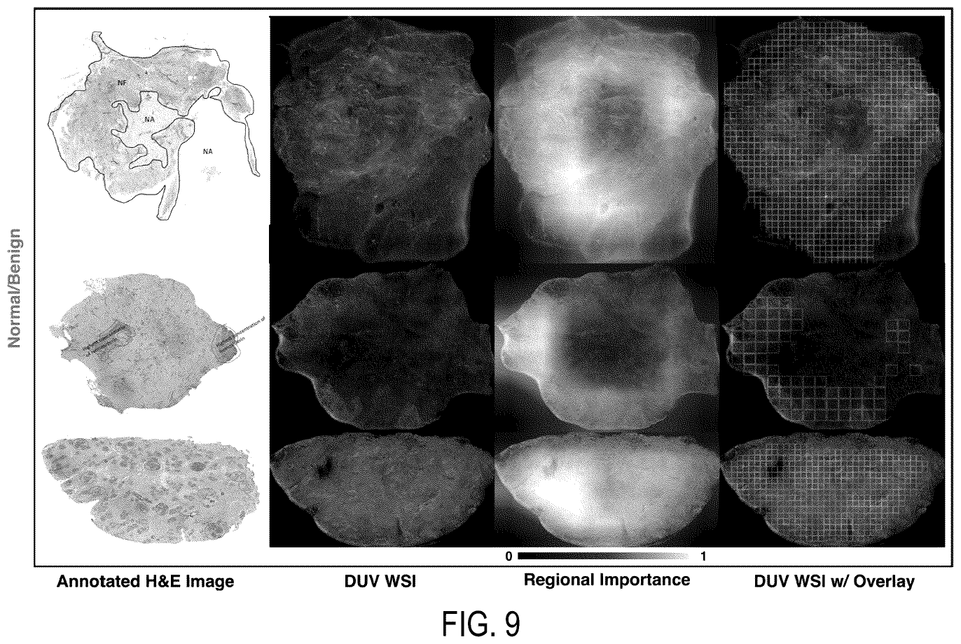

The three normal/benign DUV WSI images that were misclassified as malignant are shown in FIG. 9. The top sample contains a mixture of fat and fibrotic breast tissue. The dense, irregular, bumpy fibrotic tissue likely contributed to the misclassification. The middle sample has a pool of bleeding that probably resulted in misclassification. The bottom sample was considered normal/benign breast tissue with high fibro glandular density, indicating dense breast tissue. However, it is acknowledged that there may be some uncertainty in the sample classification due to other possible confounding factors. Thus, it is recommended to conduct additional analysis and confirmation to validate the classifications.

This study used a pre-trained ResNet50 model with ImageNet weights to extract convolutional features from DUV WSI patches, which were then used to train an XGBoost classifier for patch-level classification. The regional importance maps were generated using the Grad-CAM++ algorithm on a pre-trained DenseNet169 model with ImageNet weights to localize crucial areas in the WSI image for margin assessment. The overall WSI label was determined by fusing the patch-level classification results with the regional importance map, which enhanced classification accuracy at the WSI level. The proposed method achieved an accuracy of 95% in determining DUV WSI and displayed 100% sensitivity in detecting malignant cases. Additionally, the approach could accurately localize malignant and normal/benign tissue areas, outperforming standard deep-learning classification methods on DUV breast surgical samples.

Integrating the proposed method into surgical navigation systems can improve precision and minimize damage to healthy tissues. Investigating the method's performance on other medical imaging modalities can enhance its versatility and applicability to different diagnosis techniques, contributing to better patient outcomes.

Accurate margin assessment during breast cancer surgery is crucial for reducing breast cancer re-excision rates following breast-conserving surgery (BCS). This study addressed the challenge of locating cancerous regions in DUV whole-slide images (WSI) by proposing an automated method leveraging deep learning techniques' power. This methodology combines patch-level classification using transfer learning with regional importance maps generated through Grad-CAM++. Focusing on highly significant regions within the images assigns a malignant or normal/benign label to each WSI with increased confidence. This approach enhances the robustness of breast cancer classification in DUV WSI images, which is advantageous for accurate intra-operative margin assessment. The proposed method was evaluated on a dataset of 60 authentic DUV WSI images, and an impressive classification accuracy of 95.0% was achieved. This result indicates the potential of the proposed method for real-time assessment of margin status during breast cancer surgery and potentially other types of cancer surgeries. The approach used in this study could impact clinical decision-making significantly. It can provide real-time guidance to surgeons during operations, ensuring they make more informed decisions about resection margins. It can also help develop personalized treatment plans. Overall, this study demonstrates the effectiveness of deep learning techniques in breast cancer margin assessment using DUV WSI images. Furthermore, this method can be integrated into surgical navigation systems to visualize cancerous regions and their boundaries in real time, improving the precision of surgical interventions and minimizing damage to healthy tissues. The potential clinical applications and the possibility for further refinement of this method offer a promising direction for future research and development in medical imaging.

In an example study, the methods described above were applied for the classification of breast cancer in deep ultraviolet (DUV) WSIs. To address the challenge of limited data and enhance tumor localization, each WSI is preprocessed and segmented into smaller, non-overlapping patches. Using a transfer learning strategy, a pre-trained ViT model is fine-tuned to perform patch classification, learning discriminative features from available DUV data. An approach was adopted to enhance the interpretability of the classifier. Fine-tuning a pre-trained DenseNet-169 network on DUV WSI data and applying Grad-CAM++ generates regional saliency maps. These visually represent the model decision-making process and highlight spatial regions important to the classification task. To obtain the WSI-level classification, patch-level predictions are multiplied by their corresponding saliency map scores to compute a weighted combination. Thresholding the results through a sign function then yields a final binary determination.

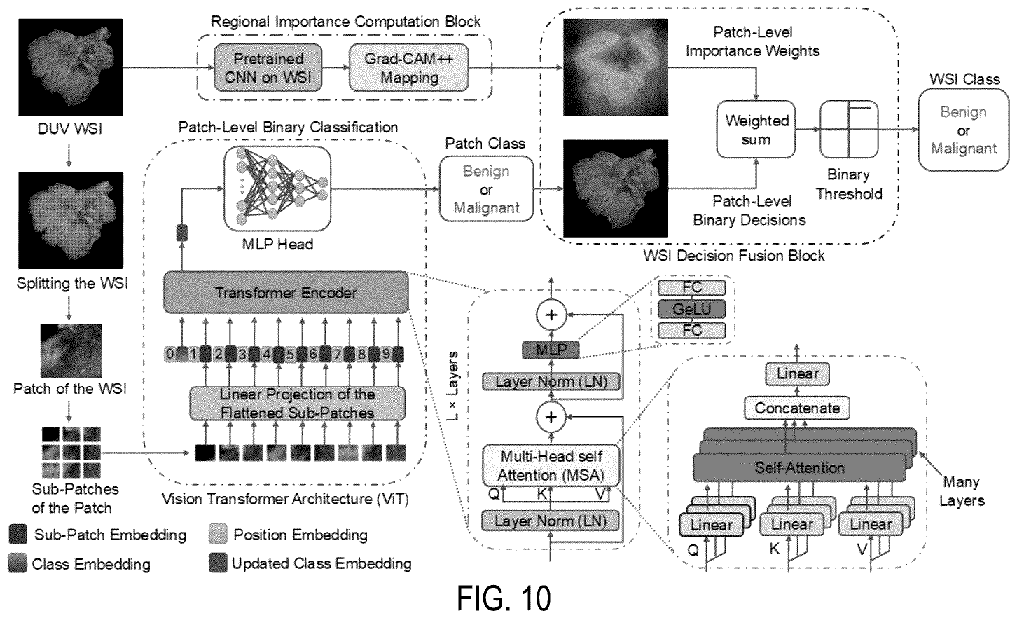

An example of the classification workflow implemented in the study is illustrated in FIG. 10. Leveraging transfer learning with a pre-trained ViT model, where common features are already understood, reduces the risk of overfitting and accelerates training [11]. After fine-tuning with DUV data, the model can extract fine-grained features and classify patches as malignant or benign.

Let xᵢ represent the i-th sample from the DUV WSI dataset, where i∈{1, . . . , M}. Partitioning each x; into smaller, non-overlapping patches of 400×400 pixels allows computational efficiency while providing sufficient detail of tissue morphology and cellular structures to make effective margin assessments. Patches containing more than 80% background are excluded. Each remaining patch

$${p}_{i}^{j}$$

becomes an individual input, being downsampled to a height H and width W to pass into the ViT-Base architecture (224×224 pixels herein). When subsequently sent through the ViT, each input

$${p}_{i}^{j}$$

gets divided into N smaller, non-overlapping sub-patches,

$${s}_{i}^{jk} ∈ {R}^{P \times P \times C} ,$$

where k ∈ {1, . . . , N} indexes the sub-patches, P×P is the sub-patch size, and C indicates the number of channels. The total number of sub-patches is given by

$$N = \frac{H \times W}{{P}^{2}} .$$

Each sub-patch

$${s}_{i}^{jk}$$

then gets flattened into a vector

$$vec ( {s}_{i}^{jk} ) ∈ {R}^{1 \times ( {P}^{2} \cdot C )}$$

and linearly projected to a fixed dimension D by means of a trainable projection matrix E∈R^{(P}^{²}^{·C)×D}. This operation is defined as:

$${\hat{s}}_{i}^{jk} = vec ( {s}_{i}^{jk} ) E .$$

Each

$${\hat{s}}_{i}^{jk}$$

represents one of N sub-patch embeddings within an input sequence. Providing an aggregate, global patch representation, a trainable class embedding

$${z}_{0}^{0} = {p}_{i}^{jclass}$$

is concatenated at the start of the sequence. A trainable positional embedding

$${E}_{i}^{jpos}$$

is then added to the sequence of embeddings, preserving patch-level location information for each sub-patch. The completed input sequence for the transformer encoder is formed by:

$${z}_{0} = [ {p}_{i}^{jclass} , {\hat{s}}_{i}^{j 1} , {\hat{s}}_{i}^{j 2} , ... , {\hat{s}}_{i}^{jN} ] + {E}_{i}^{jpos} .$$

A transformer encoder processes an input z₀ through layers [symbol] ∈{1, . . . , L} that include layer normalization (LN), multi-head self-attention (MSA), and multi-layer perceptron (MLP) blocks (two linear layers with separating GELU activation), both blocks incorporating residual connections. For each layer, the operations are defined as follows:

$$\begin{matrix}{z}_{ℓ}^{′} = M S A ( L N ( {z}_{ℓ - 1} ) ) + {z}_{ℓ - 1} \\ {z}_{ℓ} = M L P ( L N ( {z}_{ℓ}^{′} ) ) + {z}_{ℓ}^{′} .\end{matrix}$$

The resulting class token

$${z}_{L}^{0}$$

undergoes a last layer normalization and passes into a single linear layer MLP_{head} to classify the j-th patch from the i-th WSI,

$${p}_{i}^{j}$$

as follows:

$${\hat{y}}_{i}^{j} = M L {P}_{head} ( L N ( {z}_{L}^{0} ) ) .$$

The predicted

$${\hat{y}}_{i}^{j}$$

and ground-truth label

$${y}_{i}^{j}$$

for a patch

$${p}_{i}^{j}$$

use values of 0 and 1 to denote benign and malignant tissues.

Let

$${Y}_{i}^{{y}_{i}}$$

denote the output logit value for the i-th WSI sample xᵢ and class corresponding to the ground-truth label yᵢ. The logit represents the raw model output before the application of a softmax function. Let F^{q} represent the q-th of Q feature maps of a selected layer, where q∈{1, . . . , Q}. The importance weight assigned to a specific feature map F^{q} for each class yᵢ is defined through:

$${λ}_{q}^{{y}_{i}} = {\sum }_{α} {\sum }_{β} {w}_{αβ}^{{qy}_{i}} \cdot ReLU ( \frac{\partial {Y}_{i}^{{y}_{i}}}{\partial {F}_{αβ}^{q}} )$$

where the indices α and β represent a pixel location and the weighting coefficient

$${w}_{α β}^{{qy}_{i}}$$

denotes its importance. The gradient

$$\frac{\partial {Y}_{i}^{{y}_{i}}}{\partial {F}_{αβ}^{q}}$$

represents the derivative of the raw output score,

$${Y}_{i}^{{y}_{i}} ,$$

with respect to the feature map F^{q} at position (α, β). The saliency map Rᵢ for the i-th WSI sample, xᵢ, is obtained by:

$${R}_{i} = ReLU ( {\sum }_{q} {λ}_{q}^{{y}_{i}} \cdot {F}^{q} )$$

The ReLU function effectively limits considered features and pixels to those with positive contributions to class prediction, emphasizing the most informative and relevant regions.

Grad-CAM++ was applied on a pre-trained DenseNet-169 model to create saliency maps using features extracted from the batch normalization layer between the final convolutional and classification layers. Let

$${A}_{i}^{j}$$

denote the region corresponding to the j-th patch of the i-th WSI sample, xᵢ. The patch saliency score averages the saliency map values over the pixel area

$${A}_{i}^{j} ,$$

being determined through:

$${r}_{i}^{j} = \frac{1}{\vert {A}^{j} \vert} {\sum }_{( α , β ) ∈ {A}_{i}^{j}} {R}_{i , αβ} .$$

Let

$$\vert {A}_{i}^{j} \vert$$

denote the total number of pixels in the j-th patch (herein 400×400 pixels) and the saliency value at position (α, β) be represented by R_{i,αβ}. Initial ViT model predictions, with labels of 0 (benign) or 1 (malignant), for a patch

$${p}_{i}^{j}$$

are remapped to −1 and +1, then denoted as

$${\tilde{y}}_{i}^{j} .$$

Applying weighted majority voting fuses the transformed values with corresponding saliency scores. An empirical threshold

$${r}_{i}^{j} > 0.3$$

ensures that only patches with significant saliency contribute to the weighted sum. This approach weights patch-level contributions to WSI-level classification according to the determined relevance of evaluated features. Resulting non-zero values are mapped via a function sign (·), to binary values, where R−→−1 and R+→+1. Overall, the final predicted label for xᵢ is computed to be:

$${\hat{y}}_{i} = \{ \begin{matrix}sign ( \frac{1}{{N}_{i}} {\sum }_{j = 1}^{N} [ {r}_{i}^{j} \cdot {\tilde{y}}_{i}^{j} ] ) & if {r}_{i}^{j} > 0.3 \\ 0 & otherwise\end{matrix} .$$

This study developed a method to classify DUV WSI collected during breast-conserving surgery (BCS) as benign or malignant. A 5-fold cross-validation was performed to ensure a robust evaluation, with separated training and test sets. Predefined folds of WSI samples were set, with one fold reserved in each iteration for testing, 80% of the remaining for training, and the other 20% for validation.

A pre-trained ViT-Base model (ViT-B/16) was fine-tuned on patches derived from training folds, using stochastic gradient descent (SGD), a learning rate of 3×10⁻⁴, and a cosine learning rate scheduler. The model that performed best across the validation sets was selected for final evaluation on the test set. Focusing predictions on relevant regions and enhancing interpretability, Grad-CAM++ was integrated with a pre-trained and fine-tuned Densenet-169.

For comparison and Grad-CAM++ analysis, the cross-validation was repeated using a pre-trained DenseNet-169 network. This model was fine-tuned with an Adam optimizer at a learning rate of 10⁻⁴ for 30 epochs, using a 40% dropout rate to match. Another pre-trained ViT model was similarly independently tuned at a 10⁻³ learning rate and evaluated. For both, the WSIs were resized to 224×224 pixels to match input dimension requirements.

For training and evaluation, a DUV WSI dataset for breast cancer analysis, including 60 tissue samples (24 benign and 36 malignant). Therefrom, 34,468 image patches (with 400×400 pixels) were extracted, including 9,444 malignant and 25,024 benign instances, with ground-truth labels assigned according to pathologist annotations.

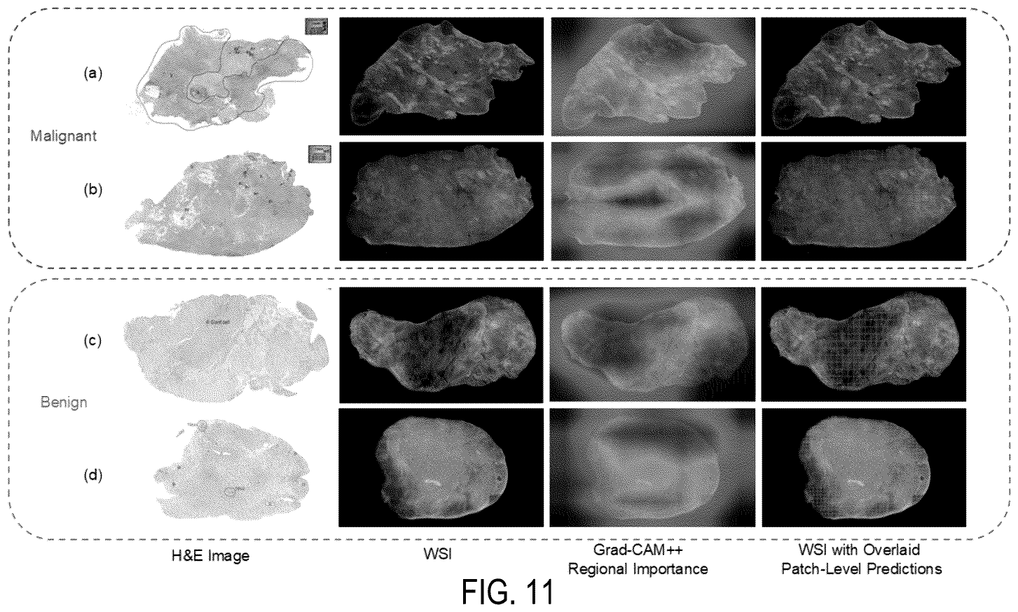

Herein are qualitative evaluations of the breast cancer detection results on malignant and benign WSIs. FIG. 11 illustrates examples from both class categories, including the corresponding H&E images with pathologist annotations, WSIs, and Grad-CAM++ saliency maps. Patch-level predictions are overlaid on the WSIs, with low-importance regions removed by an empirical threshold to emphasize the most diagnostically relevant areas. Grad-CAM++ highlights class-relevant regions with a more intense reddish hue, indicating areas where the fine-tuned DenseNet-169 model focused during classification. This decision fusion weighting mechanism was observed to prevent incorrect patch-level predictions made by the ViT, as evident in cases (b) and (c). For case (b), there were tumor cells characterized by variable cellularity within dense fibrous tissue and interspersed benign elements. Case (c) presented predominantly normal tissue with scattered small inflammatory blue cells and no definite tumor. In both cases, these misclassified patches were de-emphasized by the Grad-CAM++ weighting, correcting the WSI-level classifications.

Prior ViT, DenseNet-169, and ResNet-50 methods that were tested all demonstrated relatively poor performance. The internal downsampling operations of the CNN models lose critical contextual information. Restriction of fine-grained details of cellular structures, cancerous regions, and other components at the early stages of these architectures limited their ability to accurately distinguish benign and malignant samples. The large model sizes and relatively small dataset exacerbated overfitting issues, further degrading performance. These behaviors matched expectations since CNNs are known to struggle to capture global spatial contexts, and ViTs typically require copious quantities of data to achieve optimal performance.

In contrast, the ViT model implemented by this work processes WSI at a patch-level. Leveraging self-attention to capture local and global contextual details enables more precise, patch-level assessments. The results were then refined by fusing classifier decisions with Grad-CAM++ saliency maps, particularly effective in cases where the ViT alone struggled or made errors due to a lack of relevant features. Grad-CAM++ weighting emphasized critical regions, suppressing irrelevant or low-confidence areas. This approach achieved an impressive 98.33% accuracy, surpassing other examined deep learning methods up to approximately 13%.

Analysis of other performance metrics was conducted, including precision: the proportion of true positive predictions; sensitivity: the proportion of actual positives correctly identified; and specificity: the proportion of actual negatives correctly identified. A limitation evidenced by the prior CNN formulation was a tendency to overpredict malignancy. Modernizing to a ViT architecture has achieved gains of about 5% and 9% in precision and specificity, while maintaining perfect recall. Practically, this demonstrates improved network detection of benign cases and reduces the number of false positives during WSI classification.

Overall, the improved precision and specificity of the methods described in the present disclosure make them a more reliable tool for medical imaging. The approach offers significant clinical benefits, where patch-level assessment may provide surgeons with precise localization of cancerous material during operations. The Grad-Cam++ saliency maps additionally indicate model confidence regarding predictions, reducing the risk of unnecessary actions on incorrect positive margin-level detections or potential determination of the need for re-excision surgeries.

This study presents an automated method for classifying DUV WSIs to differentiate benign and malignant tissues during breast-conserving surgery (BCS) and accurately localize cancerous cells. Given the computational limitations of deep learning models when processing high-resolution WSIs, a patch-level framework was adopted to improve computational tractability and efficiency. Through a robust 5-fold cross-validation, it was demonstrated that vision transformer (ViT) model performance may be enhanced with Grad-CAM++ weighted decision fusion. The ViT effectively extracts both local and global features from WSI patches, while Grad-CAM++ refines patch-level predictions, leading to more accurate WSI classification, even in particularly challenging cases. The proposed method should provide valuable insights to surgeons and pathologists, reducing the risk of incorrect cancer margin detection and minimizing the need for additional surgeries.

In another example study, the methods described in the present disclosure were used to semi-automatically transfer outlines of tumor tissue from pathologist-annotated H&E images to corresponding fluorescence images. By overcoming challenges like tissue deformation, contrast differences, and resolution disparities, the disclosed methods can accelerate the validation of emerging fluorescence imaging modalities. Compared to manual annotation, the tool is significantly faster, more accurate, and cost-effective, thus shortening the cycle of developing new fluorescence imaging technologies.

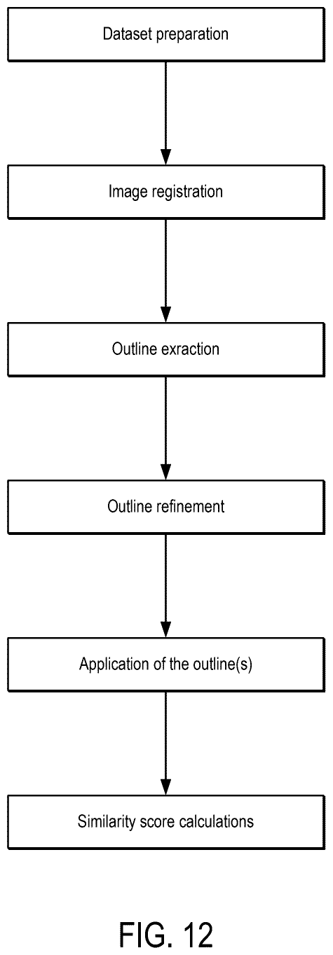

The semi-automatic annotation method includes multiple steps, including dataset preparation, image registration, outline(s) extraction from the H&E image, outline refinement, application of the outline(s) to the MUSE image, comparison annotations obtained using the semi-automated and manual method, and calculation of similarity scores between the two annotations, as illustrated in FIG. 12.

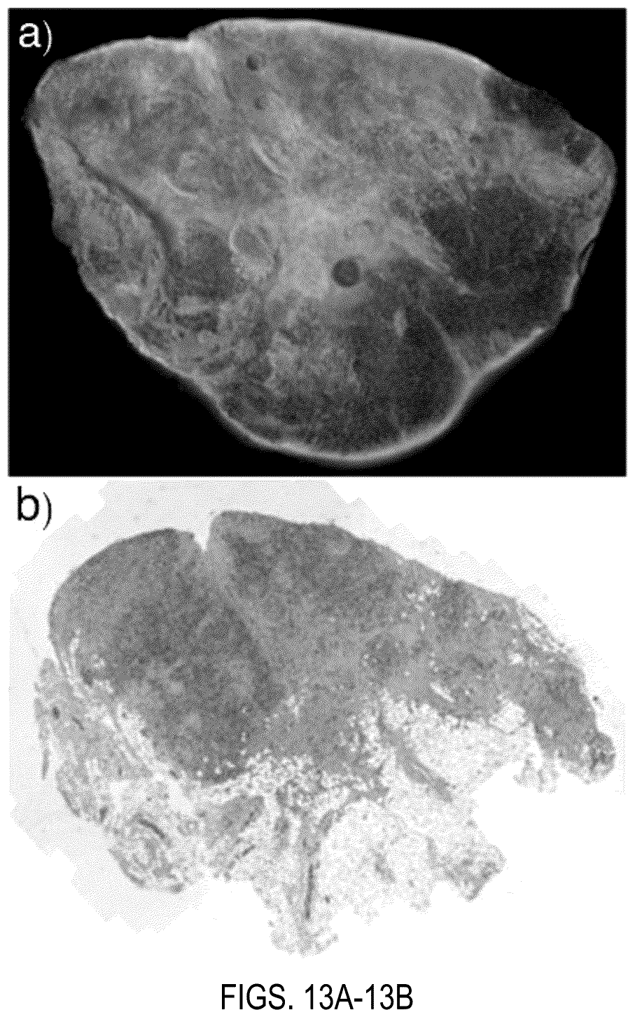

MUSE images were acquired from tissue samples using the MUSE setup in the lab. Freshly excised tissue samples were stained with fluorescence dyes, specifically propidium iodide and eosin Y, to enhance image contrast. After staining, the samples were scanned using the MUSE system to capture the fluorescence images. Following image acquisition, the tissue samples were returned to FFPE process, H&E staining, and generation of corresponding histological images for tumor annotations. MUSE images were scanned and stitched in the JPEG format, while H&E images were scanned in the MIRAX format, as shown in FIGS. 13A and 13B, respectively. The study has collected a total of 35 pairs of H&E and MUSE images from breast or lung tissue samples that contain both malignant and normal tissues.

Rigid or affine transformations like translation, rotation, or scaling may not provide accurate alignment in registration between H&E and MUSE images due to their different resolutions and local deformation. Therefore, flexible, non-rigid, or elastic registration techniques were used to align the tissue correctly in MUSE and H&E images, even though there was no guarantee of consistency in the deformation metrics. The purpose of introducing non-rigid registration is to deform local areas of MUSE images to compensate for the stretching or missing area in H&E images.

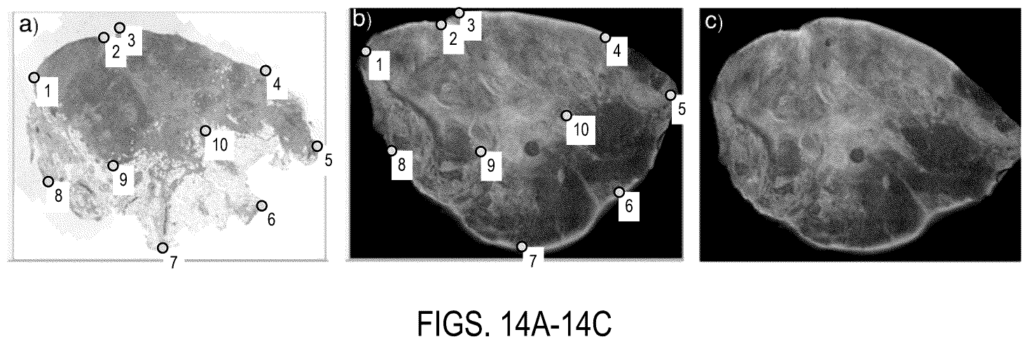

There is no standardized or fixed deformation model for the registration process because each sample might have undergone unique stretching, shrinking, or warping during the FFPE process. More importantly, the cutting depth from the tissue block can also vary, meaning that sections may not be taken from the same depth, which further complicates direct image registration. Therefore, a semi-automated registration method was introduced, which was based on manually selected corresponding paired points in H&E and MUSE images. The registration metrics varied in tissue samples, allowing for more flexibility in addressing the unpredictable deformations caused by the FFPE process and the variations in cutting depth. At least six pairs of the corresponding points from the same tissue region were clearly identified and recorded in both image modalities, with the points from the pathologist-annotated H&E image referred to as ‘fixed points’ and those from the MUSE image as ‘moving points’. FIGS. 14A and 14C show an example of selected point pairs, and FIG. 14C shows a registered MUSE image.

Next, a second-order polynomial transformation was applied to register the MUSE image to the H&E image based on these paired points, accommodating the complex, non-linear distortions in the tissue, defined as:

$$X = {a}_{0} + {a}_{1} x + {a}_{2} y + {a}_{3} {x}^{2} + {a}_{4} xy + {a}_{5} {y}^{2}$$

$$Y = {b}_{0} + {b}_{1} x + {b}_{2} y + {b}_{3} {x}^{2} + {b}_{4} xy + {b}_{5} {y}^{2}$$

where (x,y) is the pixel position in the raw MUSE image, (X,Y) is pixel position in the registered MUSE image, and a₀, a₁, . . . , a₅ and b₀, b₁, . . . , b₅ are coefficients calculated by using the selected paired points. The registered points varied across tissue samples, reflecting the inherent variability in tissue deformation during the FFPE process. Polynomial transformation was used for registration since the tumor outlines in the MUSE image were curved. A higher order of the polynomial may lead to a better fit but also requires more paired points and may result in overfitting.

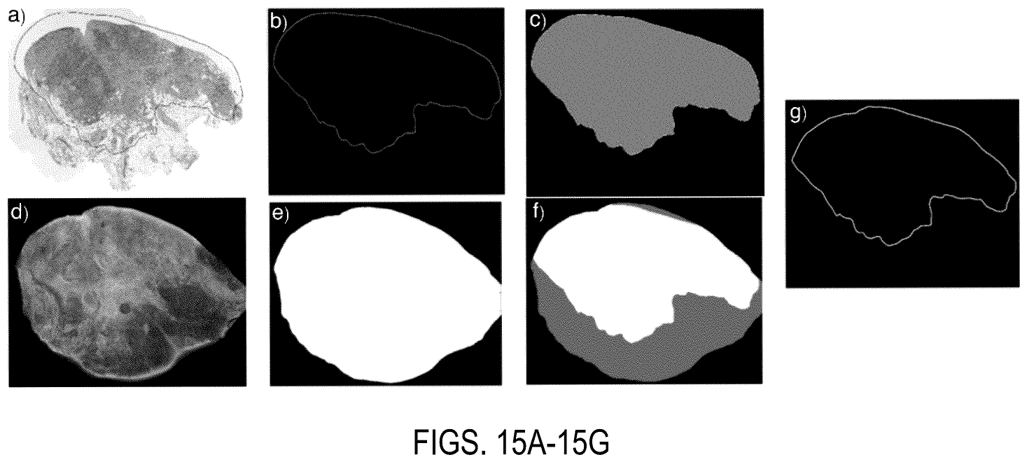

The tumor outline(s) from the pathologist-annotated H&E image was obtained by a simple subtraction arithmetic algorithm. Two files were exported from the H&E image in Case Viewer, a free slide viewing software, one with annotation from the pathologists and the other without. Next, the exact outline was obtained by subtracting the image without outline from the one with it. Because the outline was not always fully closed, the morphological structuring element was used to close and enhance it. This resulted in the first mask, named ‘annotation mask’, as illustrated in FIGS. 15A-15C.

The annotation outline extracted from the H&E images may include background regions based on pathologists' experience, as these areas could potentially contain tumor cells from deeper layers. This, however, was not applicable to MUSE images. Therefore, the Canny edge detector was performed on the registered MUSE images (FIG. 15D) to separate the tissue from the background, creating a secondary mask, referred to as the ‘tissue mask’, to refine the annotation, as shown in FIG. 15E. The refined annotation was defined as the overlapping region between the initial annotation mask and the tissue mask, as illustrated in FIG. 15F. This refined annotation as shown in FIG. 15G was then applied to the registered MUSE images to obtain an annotated MUSE image.

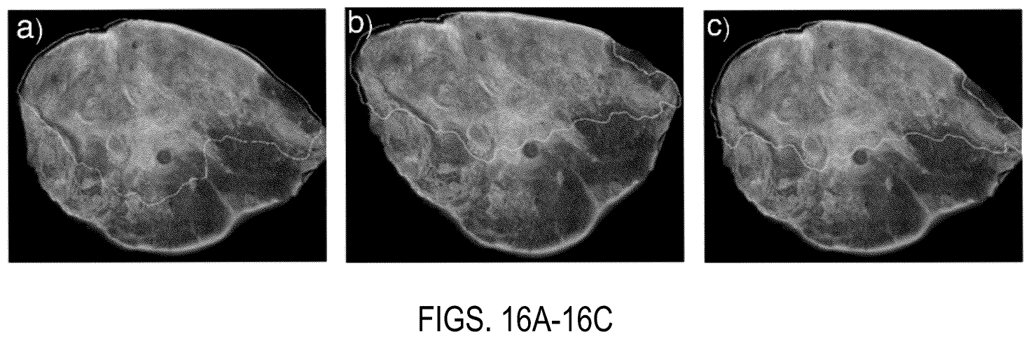

The last step was to determine the accuracy of the semi-automatic method. The ground truth to evaluate the accuracy should be based on the annotations on H&E images by the board-certified pathologist. While it is not impossible, directly comparing the tumor outline in the H&E image and that in the MUSE image obtained with the semi-automatic method can be very difficult due to the different contrasts and image shapes. Consequently, manual annotations on MUSE images, guided by the H&E annotations, were used as the gold standard to determine the performance of the method. Manual annotations were performed by a medical student and the tumor outlines in the MUSE images were confirmed by the pathologist. Because the semi-automated registered MUSE images presented slightly different contours from the corresponding manually annotated raw MUSE images without registration, the manually annotated MUSE images were registered for a fair comparison using the same metrics applied in the semi-automatic annotation process prior to the comparison, as illustrated in FIGS. 16A-16C. Three metrics, including the DICE similarity coefficient (DSC), cosine similarity, and CNN-based feature similarity, were used to evaluate the accuracy of the semi-automatic method.

The DSC, defined as the ratio of the intersection of two contours to their union, was calculated to determine the similarity of the tumor outlines in the registered MUSE image pairs obtained using the two approaches (semi-automatic vs. manual).

$$DSC = \frac{2 \times \vert {I}_{1} ⋂ {I}_{2} \vert}{\vert {I}_{1} \vert + \vert {I}_{2} \vert}$$

where I₁ and I₂ are manually and semi-automated annotated MUSE images, respectively. A DSC score of 0 indicates no overlap, while a DSC score of 1 indicates a perfect overlap, between the manually and semi-automatically obtained outlines.

Cosine similarity is a measurement that quantifies the similarity between two or more vectors, which could be applied to the MUSE images. The cosine similarity is the cosine of the angle between vectors.

$$cos ( θ ) = \frac{{I}_{1} \cdot {I}_{2}}{‖ {I}_{1} ‖ {I}_{2} ‖} = \frac{{\sum }_{i = 1}^{n} {I}_{1 i} {I}_{2 i}}{\sqrt{{\sum }_{i = 1}^{n} {I}_{1 i}^{2}} \sqrt{{\sum }_{i = 1}^{n} {I}_{2 i}^{2}}}$$

Here I₁ᵢ and I₂ᵢ represent the pixel indices of the manually and semi-automatically annotated MUSE images, respectively. A higher value indicates higher similarity. Cosine similarity was employed to assess the alignment of the tumor annotations in terms of angular similarity, focusing on the structural features of the annotations.

This metric calculates the cosine similarity between features extracted by a pretrained AlexNet convolutional neural network (CNN). AlexNet has been trained on over a million images and is capable of classifying images into 1,000 object categories and allows to compare annotations based on the high-level content while ignoring irrelevant factors like scale and intensity variations. The network has learned rich feature representations from a diverse range of images, making it highly effective at capturing meaningful patterns. This capability enables it to serve as an additional metric for evaluating the similarity between manual and semi-automatic tumor annotations.

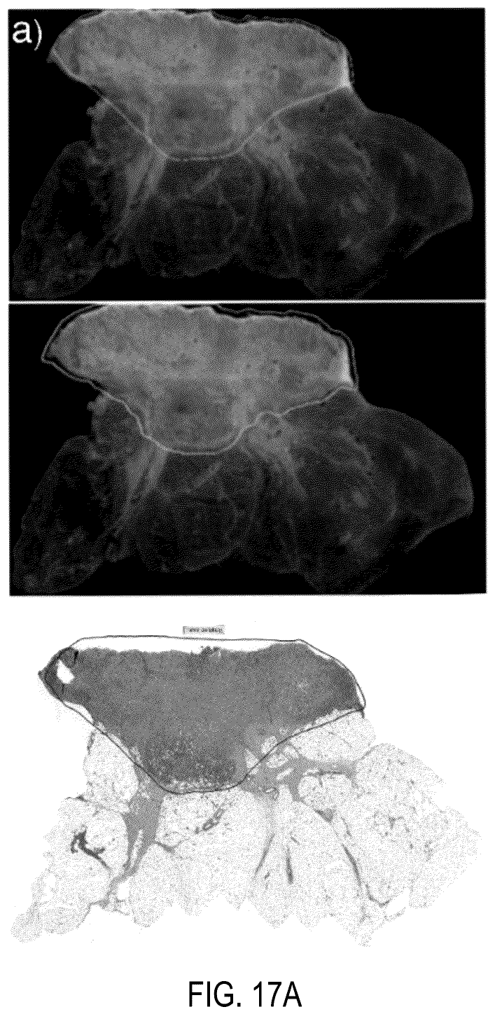

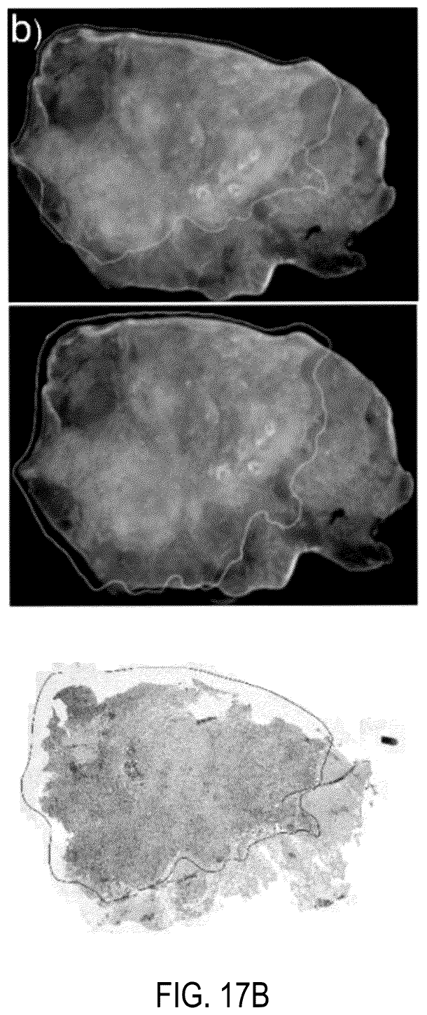

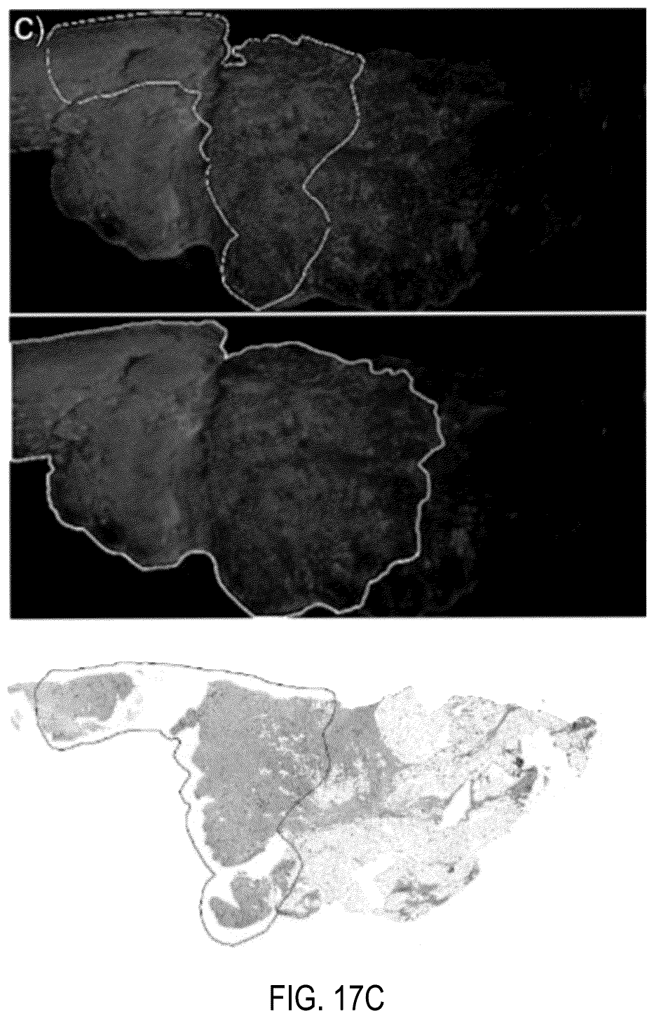

The semi-automatic annotation approach was utilized to transfer the tumor annotations from the pathologist annotated H&E images of 35 tissue samples to the corresponding registered MUSE images. The similarity scores were calculated using the DSC, cosine similarity, and CNN-based feature similarity methods. The annotated MUSE images of three representative samples with the best, medium and worst performances are presented in FIGS. 17A-17C.

The DSC for all samples exhibited an average score of 0.88 across all samples, highlighting substantial consistency in annotation boundaries between the two methods. Similarly, cosine similarity, which measures the alignment of structural orientation, also achieved an average score of 0.88, confirming the high concordance between the two annotation sets. The CNN-based feature similarity, which captures high-level feature correspondence, yielded the highest average score of 0.94, underscoring the robustness of the semi-automated approach in tumor annotation transfer. Further, the DSC ranged from 0.61 to 0.96 with the interquartile range (IQR) spanning from 0.85 (25th percentile) to 0.93 (75th percentile), with a median value of 0.90 and a standard deviation of 0.08, demonstrating a reliable performance across the samples. Cosine similarity scores varied between 0.53 and 0.98 with the IQR from 0.85 to 0.95, with a median value of 0.92 and a standard deviation of 0.11, indicating a strong structural alignment in most cases. The CNN-based feature similarity scores ranged from 0.85 to 0.99 with the IQR from 0.91 to 0.97, with a median value of 0.95 and a standard deviation of 0.04, further validating the consistency of the method. These results collectively demonstrate strong agreement between the semi-automated and manual annotations, suggesting that the semi-automated approach performs well in transferring tumor annotations from H&E to MUSE images.

DL-based MUSE imaging demonstrated great potential for intraoperative assessment of tumor margins but requires a large number of annotated images for model training and testing. The developed semi-automatic method aimed to transfer annotations accurately and efficiently from pathologist-annotated H&E images to MUSE images, thus facilitating the development of DL algorithms for intraoperative tumor classification. The results indicated a robust overall performance, with median values for all three similarity metrics, including DSC, cosine similarity, and CNN-based feature similarity, approaching or exceeding 90%, underscoring the reliability of the method. The low standard deviation across these metrics further highlights its consistency across diverse samples.

This semi-automated method represents a novel approach for transferring tumor annotations from histological H&E images to fluorescence images captured by MUSE, utilizing colored images rather than grayscale images. The disclosed method benefits from leveraging whole-tissue, high-resolution fluorescence images generated by MUSE, enabling more accurate and contextually relevant annotation transfer. Moreover, this method can be applied to colored images, in contrast to the grayscale images typically used in existing tools. This improvement allows for the preservation of richer color-based features, which are particularly important in fluorescence imaging.

While whole slide imaging has been widely used in digital pathology, the integration of fluorescence images for intraoperative decision-making remains relatively underexplored. Prior methods still solely rely on H&E images as the gold standard for validating their effectiveness. The disclosed method enhances this process by facilitating the registration of different imaging modalities and enabling the direct transfer of annotations, which offers a more efficient and streamlined method for mapping H&E annotations into different imaging modalities. By facilitating rapid, real-time annotation transfer across whole tissue images, the disclosed method is poised to significantly improve the accuracy and speed in developing intraoperative tumor classification algorithms, providing a valuable tool for real-time surgical decision-making.

The semi-automatic annotation transfer method effectively transferred tumor annotations from pathologist-annotated H&E images to MUSE images with excellent DSC, cosine similarity, and CNN-based feature similarity scores, demonstrating its potential in accelerating the development of DL-based MUSE imaging for improving intraoperative assessment of tumor margins. The method may be readily extended for annotating other fluorescence images but requires validation with relevant images.

The present disclosure has described one or more preferred embodiments, and it should be appreciated that many equivalents, alternatives, variations, and modifications, aside from those expressly stated, are possible and within the scope of the invention.

1. A scanning microscopy system, comprising:
a sample holder to contain a tissue sample;
a first imaging apparatus arranged on a first side of the sample holder, comprising:
a first ultraviolet light source to illuminate the first side of the sample holder;
a first camera to receive light emitted from the tissue sample from the first side of the sample holder;
a second imaging apparatus arranged on a second side of the sample holder that is opposite the first side, comprising:
a second ultraviolet light source to illuminate the second side of the sample holder; and
a second camera to receive light emitted from the tissue sample from the second side of the sample holder.

2. The scanning microscopy system of claim 1, further comprising a computer system to:
receive first image data from the first camera and second image data from the second camera; and
output one or more images of the sample from the first image data and the second image data.

3. The scanning microscopy system of claim 1, wherein the sample holder comprises an optically transparent box having a moveable plate to compress the sample to fill a volume of the box.

4. The scanning microscopy system of claim 3, wherein the optically transparent box is composed of quartz.

5. The scanning microscopy system of claim 1, further comprising an optical camera to acquire an optical image of the tissue sample.

6. The scanning microscopy system of claim 5, further comprising a computer system to:
receive the optical image from the optical camera;
determine an imaging area on the tissue sample from the optical image; and
direct the first imaging apparatus and second imaging apparatus to acquire first imaging data and second imaging data, respectively, in parallel from the tissue sample by scanning over the determined imaging area.

7. The scanning microscopy system of claim 6, wherein the computer system also determines an initial imaging point from the optical image and directs the first imaging apparatus and second imaging apparatus to scan over the determining imaging area starting at the initial imaging point.

8. A method for deep-ultraviolet scanning microscopy, comprising:
acquiring first image data from a sample by:
illuminating a first side of the sample with a first ultraviolet light source;
detecting light emitted from the first side of the sample using a first camera;
acquiring second image data from the sample by:
illuminating a second side of the sample with a second ultraviolet light source;
detecting light emitted from the second side of the sample using a second camera; and
outputting at least one image of the sample from the first image data and the second image data.

9. The method of claim 8, wherein the first image data and the second image data comprise images that include a combination of intrinsic and extrinsic fluorescent signals.

10. The method of claim 9, wherein the intrinsic fluorescent signals comprise fluorescent signals from fluorescent light emitted from tryptophan.

11. The method of claim 9, wherein the extrinsic fluorescent signals comprise fluorescent signals from fluorescent light emitted from at least one fluorophore.

12. The method of claim 11, wherein the at least one fluorophore comprises propidium iodide or eosin Y.

13. The method of claim 11, wherein the at least one fluorophore comprises both propidium iodide and eosin Y.

14. The method of claim 8, wherein the first image data and the second image data are acquired in parallel.

15. The method of claim 8, wherein the first image data and the second image data are acquired by sparsely sampling the sample.

16. The method of claim 8, further comprising analyzing the at least one image by inputting the at least one image to a machine learning model that has been trained on training data to generate classified feature data indicating whether cancer cells are present on the sample.

17. The method of claim 16, further comprising:
dividing the at least one image into a plurality of patches;
extracting texture features from each patch; and
classifying each patch as tumor tissue or normal tissue using a classifier trained on the extracted texture features.

18. The method of claim 17, wherein the texture features are extracted from each patch using a local binary pattern algorithm.

19. The method of claim 18, wherein the local binary pattern algorithm uses a uniform rotation-invariant configuration with a number of neighboring pixels at a distance from a central pixel.

20. A method for automated classification of deep ultraviolet fluorescence images for tumor margin assessment, comprising:
dividing a deep ultraviolet fluorescence whole slide image of a tissue specimen into a plurality of patches;
extracting features from each patch using a first pre-trained convolutional neural network;
classifying each patch as tumor tissue or normal tissue using a classifier trained on the extracted features;
generating a regional importance map for the whole slide image using a visual explanation process applied to a second pre-trained convolutional neural network; and
determining a whole slide image classification by fusing patch-level classifications with the regional importance map through a weighted decision fusion.

21. The method of claim 20, wherein the visual explanation process comprises a Grad-CAM++ process.

22. The method of claim 20, wherein the first pre-trained convolutional neural network used for extracting features is a ResNet50 model.

23. The method of claim 20, wherein the second pre-trained convolutional neural network is a DenseNet169 model.

24. The method of claim 20, wherein the visual explanation process is applied to features extracted from a batch normalization layer between a final convolutional layer and a classification layer of the second pre-trained convolutional neural network.

25. The method of claim 20, wherein the classifier trained on the extracted features is an XGBoost classifier.

26. The method of claim 20, wherein the weighted decision fusion applies a threshold to regional importance values to exclude patches with low importance from the whole slide image classification.

27. The method of claim 26, wherein the threshold excludes patches having regional importance values below 0.25.

28. A method for semi-automated transfer of tumor annotations from an annotated image to an unannotated image, comprising:
obtaining the annotated image of a tissue specimen captured using a first imaging modality;
obtaining the unannotated image of the tissue specimen captured using a second imaging modality that is different from the first imaging modality, wherein the annotated image is a different image type than the unannotated image;
registering the unannotated image to the annotated image using a transformation based on corresponding point pairs selected between the annotated image and the unannotated image;
extracting tumor annotation outlines from the annotated image;
refining the extracted annotation outlines by applying edge detection to the registered unannotated image to create a tissue mask and determining an overlap between the annotation outlines and the tissue mask; and
transferring the refined annotation outlines to the registered unannotated image.

29. The method of claim 28, wherein the annotated image comprises a whole slide image.

30. The method of claim 29, wherein the whole slide image comprises a hematoxylin and eosin stained image.

31. The method of claim 30, wherein the unannotated image comprises a fluorescence image acquired using deep-ultraviolet scanning microscopy (DDSM).

32. The method of claim 28, wherein the transformation used to register the unannotated image to the annotated image comprises a second-order polynomial transformation.

33. The method of claim 28, wherein at least six pairs of corresponding points are selected from both the annotated image and the unannotated image to determine transformation coefficients for the transformation.

34. The method of claim 28, wherein the extracted annotation outlines are enhanced using morphological structuring elements to close the outlines.

35. The method of claim 28, wherein the tissue mask created by edge detection separates tissue regions from background areas in the registered unannotated image.

36. The method of claim 35, wherein the refined annotation outlines are obtained by computing an intersection between the extracted annotation outlines and the tissue mask to eliminate background regions inadvertently included in manual annotations.
